(19) United States
(12) Patent Application Publication
(10) Pub. No.: US 2026/0345663 A1
(43) Pub. Date: Nov. 5, 2026

(54) DEFIBROTIDE FOR USE IN PREVENTING AND TREATING SEPSIS

(71) Applicant: JAZZ PHARMACEUTICALS IRELAND LIMITED, Dublin (IE)

(72) Inventors: Ju SHI, Mountain View, CA (US); Polly Rae PINE, Dillon, CO (US); Sacha Sergio ZEERLEDER, Bern (CH)

(21) Appl. No.: 19/123,601

(22) PCT Filed: Oct. 24, 2023
(86) PCT No.: PCT/US2023/077630

§ 371 Date: Apr. 23, 2025

Related U.S. Application Data
(60) Provisional application No. 63/419,075, filed on Oct. 25, 2022.

(51) Int. Cl. A61K 31/711; A61K 9/00; A61P 31/04

(52) U.S. Cl. CPC A61K 31/711; A61K 9/0019; A61P 31/04

(57) ABSTRACT

The present disclosure provides methods of preventing, lessening the effects, or treating sepsis comprising administering a prophylactically effective amount of defibrotide to a subject in need thereof.

### CROSS REFERENCE

The present application claims priority to and benefit from U.S. Provisional Application No. 63/419,075 filed Oct. 25, 2022, the entire contents of which are herein incorporated by reference.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

This application incorporates by reference the following publications, patents and applications in their entireties for all purposes: U.S. application Ser. Nos. 16/105,319 filed Aug. 3, 2018; 62/776,500 filed Dec. 7, 2018 and 62/802,099 filed Feb. 6, 2019 and 62/753,711, filed on Oct. 31, 2018 and Ser. No. 16/398,978, filed on Apr. 30, 2019 and 63/011,645 filed Apr. 17, 2020; as well as U.S. Pat. Nos. 3,770,720, 3,829,567, 3,899,481, 4,649,134, 4,693,995,4,938,873, 4,985,552, 5,081,109, 5,116,617, 5,223,609, 5,646,127, 5,646,268, and 6,046,172.

### FIELD

The present disclosure is directed to methods of administering defibrotide to prevent and/or treat sepsis.

### BACKGROUND

Sepsis is a worldwide challenge for the public healthcare system that affects over 30 million people each year, and causes over 5 million death annually (Fleischmann et al. 2016). Sepsis is a life-threatening organ dysfunction caused by a host's dysfunctional immune response to infection affecting innate and adaptive immunity, respectively (David et Brunkhorst 2017). The activation of the innate immune system is triggered by pattern-recognition receptors (PRRs) upon ligation by pathogen-associated molecular patterns (PAMPS) and damage-associated molecular patterns (DAMPs). Prominent PAMPs released from bacteria are Lipopolysaccharide (LPS) which is contained in outer membrane of gram-negative bacteria like Escherichia coli(E. coli), and lipoteichoic acid (LTA) as a part of cell membrane of most gram-positive bacteria like Streptococcus pneumonia (S. pneumonia). These two are among the bacteria most frequently causing sepsis. Cell damage in sepsis and trauma results in a local and systemic release of DAMPs such as: RNA, high mobility group box (HMGB), heat shock proteins, S100 proteins, hyaluronic acid degradation products, host DNA fragments from the nucleus (e.g. nucleosomes) or mitochondria, histones (Denning et al. 2019; Huang, Cai, et Su 2019; Raymond et al. 2017). Neutrophil extracellular traps (NETs) play a crucial role in the innate host immune response in sepsis. NETs are web-like extracellular structures consisting of genomic DNA and DNA-binding neutrophilic proteins, including histones, myeloperoxidase and neutrophil elastase, expelled by neutrophils upon activation neutrophils. The NETs degrade virulence factors and kill bacteria (Brinkmann et al. 2004) virus and fungus pathogens (Schönrich et Raftery 2016; Niedźwiedzka-Rystwej et al. 2019). Nevertheless, NETs may cause collateral damage by inducing cell death (Saffarzadeh et al. 2012), promote vaso-occlusion/thrombosis, modulate sterile inflammation and promote tumor growth and metastasis (Papayannopoulos 2018).

It has been shown that increased markers for NET formation were found in patients suffering from systemic inflammation: circulating DNA in the form of nucleosomes and neutrophil activation were significantly increased in sepsis patients and significantly correlated with severity and outcome (Zeerleder et al. 2003; Zeerleder et al. 2003). In addition, in children suffering from meningococcal sepsis nucleosome levels correlated with the inflammatory response severity and were associated with mortality (Zeerleder et al. 2012). Histones have been demonstrated to mediate lethality in sepsis and neutralization of histones by monoclonal antibodies rescues animal to die from sepsis (Xu et al. 2009). Thus, prophylactic or therapeutic strategies against the cytotoxicity activity of nucleosomes or histones may be promising.

At the time of this disclosure, most of clinical trials for sepsis therapies failed. Thus, there is a need for the development of new strategies to prevent and treat sepsis.

### SUMMARY OF THE INVENTION

The present disclosure provides prophylactic protocols for the prevention, treatment, management or amelioration of sepsis, or other dysregulated inflammatory responses. In some embodiments, the present disclosure provides methods of preventing or treating sepsis comprising administering a prophylactically effective amount of defibrotide to a subject in need thereof.

In some embodiments, the present disclosure provides methods of preventing or treating sepsis comprising administering a prophylactically effective amount of defibrotide to a subject with an infection before the onset of sepsis or symptoms thereof.

In some embodiments, the present disclosure provides methods of preventing or treating sepsis comprising administering a prophylactically effective amount of defibrotide to a subject who is at high-risk for developing sepsis. In some embodiments, the subject who is considered at high-risk for developing sepsis is selected from a group consisting of an elder adult 65 or older, an infant younger than one, a person with a chronic condition, a person with a weakened immune system, and a pregnant woman. In some embodiments, a chronic condition comprises diabetes, cancer, lung disease, kidney disease and liver disease.

In some embodiments, the present disclosure provides methods of preventing or treating sepsis comprising administering a prophylactically effective amount of defibrotide to a subject when an altered level of a biomarker associated with sepsis is detected in the subject before the onset of sepsis or symptoms thereof. In some embodiments, the biomarker is selected from a group consisting of IFNγ, IL-1α, IL-1β, RANTES, MIP-2 and PAI-1.

In some embodiments, the defibrotide is administered prophylactically to a subject at risk of developing sepsis after administration of another therapy. In some embodiments, the prior therapy may comprise antibiotic or corticosteroid therapy.

In some embodiments, the defibrotide is administered at a dose between 1 mg/kg and 40 mg/kg. In some embodiments, the defibrotide is administered at a dose of 20 mg/kg.

In some embodiments, the defibrotide is administered once a day. In some embodiments, the defibrotide is administered in multiple doses per day. In some embodiments, the defibrotide is administered in two to ten doses per day. In some embodiments, the defibrotide is administered four times a day. In some embodiments, the defibrotide is administered every six hours. In some embodiments, the defibrotide is administered by continuous infusion, subcutaneous delivery or intravenous delivery.

In some embodiments, the defibrotide is administered by four consecutive two-hour intravenous infusion at a dose of 20 mg/kg, six hours apart.

### BRIEF DESCRIPTION OF THE DRAWINGS

FIGS. 1A-1E illustrates the effects of defibrotide on survival rate and clinical feature. FIG. 1A delineates the protocol designed of LPS-induced sepsis in mouse treated or not with defibrotide (DF); FIG. 1B shows the pilot survival rate study results to establish 50 lethality dose (LD50) with different LPS doses: 5 mg/kg, 10 mg/kg, 20 mg/kg and 30 mg/kg by i.v. injection, n=5 for NaCl group and n=8 for all LPS doses groups, ****p<0.0001 for all the graph, ***p<0.001 NaCl group versus LPS at 30 mg/kg group, *p<0.05 NaCl group versus LPS at 20 mg/kg determined with Mantel-Cox test; FIG. 1C shows the survival rate during defibrotide treatment at different doses 25 mg/kg/day (low) in prophylactic or therapeutic injection, 175 mg/kg/day (medium) and 350 mg/kg/day (high) in therapy, n=6 for all NaCl treated and untreated groups and LPS treated with DF at 175mg/kg/day and 350 mg/kg/day, n=12 for LPS untreated group and LPS treated in prophylaxis with DF at 25 mg/kg/day, n=14 for LPS treated with DF at 25 mg/kg/day, *p<0.05 LPS groups versus NaCl groups determined with Mantel-Cox test; FIG. 1D shows the cumulative score based on clinical features of mice during DF treatment after LPS challenge; FIG. 1E shows body weight loss in mice for 4 days with or without DF treatment followed after LPS challenge. n=6 for all NaCl treated and untreated groups, n=8 for all LPS treated and untreated groups.

FIGS. 2A-2D illustrate the measurements of specific markers for organ injury in plasma at different times point: 2 hours, 14 hours and 24 hours after LPS challenge in mice untreated or treated with defibrotide (DF) as a prophylactic treatment at low dose corresponding to 25 mg/kg/day, or as therapeutic treatment at low or medium dose at (25 mg/kg/day and 175 mg/kg/day respectively). A. Lactate dehydrogenase (LDH); B. Aspartate aminotransferase (AST); C. Urea; D. Creatinine. For all graphs, n=6-8 for all LPS groups treated or untreated and for all NaCl groups treated; n=4-6 for all NaCl groups untreated. The statistical significance was determined with Kruskal-Wallis test then Dunn's multiple comparison test or Mann-Whitney test: *p<0.05, **p<0.01.

FIGS. 3A-3F illustrate the effect of defibrotide on the inflammatory response in spleen. Cytokines/chemokines were measured in the spleen at different times point: 2 hours, 14 hours and 24 hours after LPS challenge in mice untreated or treated with defibrotide (DF) in prophylaxis at low dose corresponding to 25 mg/kg/day, or in therapy at low dose or medium dose at (25 mg/kg/day and 175 mg/kg/day respectively). A. Spleen's weight; B. TNF-α; C. IL- 1β; D. IL-6; E. Chemokine KC/CXCL1; F. Myeloperoxidase (MPO). The different parameters were measured in plasma at different times point: 2 hours, 14 hours and 24 hours after LPS challenge. For all graphs, n=6-8 for all LPS groups treated or untreated and for all NaCl groups treated; n=4-6 for all NaCl groups untreated. The statistical significance was determined with Kruskal-Wallis test then Dunn's multiple comparison test: *p<0.05, **p<0.01, ***p<0.001, ****p<0.0001.

FIGS. 4A-4F illustrate the effect of defibrotide on inflammatory response in lungs and liver. Different cytokines/chemokines were measured in lungs and liver at different times point: 2 hours, 14 hours and 24 hours after LPS challenge in mice untreated or treated with defibrotide (DF) in prophylaxis at low dose corresponding to 25 mg/kg/day, or in therapy at low dose or medium dose at (25 mg/kg/day and 175 mg/kg/day respectively). A. Lungs weight; B. Liver weight; C. TNF-α in lung; D. TNF-α in liver; E. IL-1β in lung; F. IL-1β in liver. For all graphs, n=6-8 for all LPS groups treated or untreated and for all NaCl groups treated; n=4-6 for all NaCl groups untreated. The statistical significance was determined with Kruskal-Wallis test then Dunn's multiple comparison test. ns: not significant; * define a comparison between LPS and NaCl groups from the same untreated or treated group at the same time point: *p<0.05, **p<0.01, ***<0.001, p=0.0001 or by Mann-Whitney test *p<0.05, **p<0.01, ***<0.001.

FIG. 5 illustrates the effect of defibrotide on nucleosome release in blood. Nucleosomes were measured in plasma at different times point: 2 hours, 14 hours and 24 hours after LPS challenge in mice untreated or treated with defibrotide (DF) in prophylaxis at low dose corresponding to 25 mg/kg/day, or in therapy at low dose or medium dose (25 mg/kg/day and 175 mg/kg/day respectively). A. nucleosome. For all graphs, n=6-8 for all LPS groups treated or untreated and for all NaCl groups treated; n=4-6 for all NaCl groups untreated. The statistical significance was determined with Kruskal-Wallis test then Dunn's multiple comparison test: *p<0.05, **p<0.01, or by Mann-Whitney test *p<0.05.

FIG. 6 shows the survival effect of defibrotide on LPS-induced mortality in rats.

FIGS. 7A-7E shows plasma cytokines and chemokine expression (mean±SD) of RANTES (FIG. 7A), IL-1α (FIG. 7B), IL-1β (FIG. 7C), IFNγ (FIG. 7D), and MIP-2 (FIG. 7E) after LPS administration versus time profiles following saline and defibrotide 2-hour iv infusion 20 mg/kg, 40 mg/kg and 80 mg/kg/dose in LPS-challenged rats, control, and defibrotide iv 2-hour infusion 80 mg/kg iv in rats not challenged with LPS.

FIG. 8 plasma biomarker expression (mean±SD) of PAI-1 after LPS administration versus time profiles following saline and defibrotide 2-hour iv infusion 20 mg/kg, 40 mg/kg and 80 mg/kg/dose in LPS-challenged rats, control, and defibrotide 2-hour iv infusion 80 mg/kg in rats not treated with LPS.

FIG. 9 shows mean defibrotide plasma concentration (±SD) versus time profiles following 1^{st} defibrotide iv 2-hour infusion 20 mg/kg, 40 mg/kg and 80 mg/kg dose in LPS treated rats, and single defibrotide iv 2-hour infusion 80 mg/kg iv in rats not treated with LPS (linear scale).

### DETAILED DISCLOSURE

Defibrotide is approved as Defitelio® (Gentium S.r.l; Jazz Pharmaceuticals) for the treatment of adult and pediatric patients with hepatic veno-occlusive disease (VOD), also known as sinusoidal obstruction syndrome (SOS), with renal or pulmonary dysfunction following hematopoietic stem-cell transplantation (HSCT). Defibrotide reduces endothelial cell (EC) activation and damage by mechanisms that are antithrombotic, fibrinolytic, anti-adhesive, and anti-inflammatory; thereby restoring the thrombotic-fibrinolytic balance and preserving endothelial homeostasis (Coccheri 1988; Cella 2001; Falanga 2003; Corbacioglu 2004; Benimetskaya 2008; Echart 2009; Palmer 1993; Pescador 2013; Richsepsison 2018). Though the mechanism of action of defibrotide has not been fully elucidated, in vitro and/or in vivo studies indicate that defibrotide increases systemic tissue factor pathway inhibitor (TFPI), tissue plasminogen activator (t-PA) expression, and thrombomodulin expression; decreases von Willebrand factor (vWF) and plasminogen activator inhibitor-1 (PAI-1) expression; and enhances enzymatic activity of plasmin to hydrolyse fibrin clots (Cella 2001; Coccheri 1988; Coccheri and Nazzari 1996; Cohen 1989; Zhou 1994; Falanga 2003; Echart 2009; Umemura 2016; Kaleelrahman 2003). In vitro, defibrotide inhibits leukocyte adhesion to endothelium by suppressing P-selectin and vascular cell adhesion molecule-1 (VCAM)-1 and interfering with lymphocyte function-associated antigen 1-intercell adhesion molecule (LFA-1-ICAM)-mediated leukocyte transmigration. Platelet adhesion is inhibited via increases in nitric oxide (NO), prostaglandin I2 (PGI2), and prostaglandin E2 (PGE2) (Biagi 1991; Ferraresso 1993; Palomo 2016). In vitro, defibrotide demonstrates anti-inflammatory effects that attenuate the release and production of reactive oxygen species and inflammatory mediators such as interleukin (IL)-1, IL-6, thromboxane A2, leukotriene B4, and tumor necrosis factor-α (TNF-α) (Ferraresso 1993; Bracht and Schrör 1994; Palomo 2011; Yakushijin 2019). Additionally, defibrotide inhibits the expression of heparanase, thereby contributing to extracellular matrix integrity (Eissner 2002; Barash 2018).

The investigator believes that through its role to stabilize the endothelium, defibrotide prevents pulmonary microthrombi, decreases pulmonary endothelial production of inflammatory cytokines, promotes vaso-dilation (increased production of NO, prostanoids), inhibits platelet activation (reduction in vWF), and/or regulates the fibrinolytic pathway (reduction in PAI-1), to lead to improvement in oxygenation and promote the resolution of SEPSIS.

### DEFINITIONS

The term defibrotide identifies a polydeoxyribonucleotide that is obtained by extraction from animal and/or vegetable tissues but which may also be produced synthetically; the polydeoxyribonucleotide is normally used in the form of an alkali-metal salt, generally a sodium salt, and generally has a molecular weight of 13 to 30 kDa (CAS Registry Number: 83712-60-1). Preferably, defibrotide is obtained according to U.S. Pat. Nos. 4,985,552 and 5,223,609 and/or presents the physical/chemical characteristics described in the same U.S. Pat. Nos. 4,985,552 and 5,223,609, herein incorporated by reference. More in particular, defibrotide is a mixture of polydeoxyribonucleotides having formula of random sequence: P1-5, (dAP)₁₂₋₂₄, (dGP)₁₀₋₂₀, (dPp)₁₃₋₂₆, (dCP)₁₀₋₂₀, where: P=phosphoric radical; dAp-deoxyadenylic monomer; dGp-deoxyguanylic monomer; dTp-deoxythymidinic monomer; dCp-deoxycytidynic monomer; and/or shows the following chemical/physical characteristics: electrophoresis=homogeneous anodic mobility, and/or extinction coefficient, E₁cm^{1%} at 260±1 nm=220±10, and/or E₂₃₀/E₂₆₀=0.45±0.04, and/or coefficient of molar extinction (referred to phosphorous) ε(P)-7.750±500, and/or rotatory power [α]_{D}^{20°}=53°±6; and/or reversible hyperchromicity, indicated as % in native DNA and/or h=15±5. In some embodiments, the defibrotide is Defitelio®.

The term “subject” is used interchangeably herein with “patient” to refer to an individual to be treated. The subject is a mammal (e.g., human, non-human primate, rat, mouse, cow, horse, pig, sheep, goat, dog, cat, etc.). The subject can be a clinical patient, a clinical trial volunteer, an experimental animal, etc. The subject can be suspected of having or at risk for having a condition or be diagnosed with a condition that leads to sepsis. The subject can also be suspected of having or at risk for having sepsis. According to one embodiment, the subject to be treated according to this invention is a human. Subjects to be treated by the methods of the disclosed embodiments include both human subjects and animal subjects (e.g., dog, cat, monkey, chimpanzee, and/or the like) for veterinary purposes. The subjects may be male or female and may be any suitable age, e.g., neonatal, infant, juvenile, adolescent, adult, or geriatric. In some embodiments, the subjects are preferably mammalian.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “effective amount,” as used herein, refers broadly to the amount of a compound, antibody, antigen, or cells that, when administered to a patient for treating a disease, is sufficient to effect such treatment for the disease. The effective amount may be an amount effective for prophylaxis, and/or an amount effective for prevention. The effective amount may be an amount effective to reduce, an amount effective to prevent the incidence of signs/symptoms, to reduce the severity of the incidence of signs/symptoms, to eliminate the incidence of signs/symptoms, to slow the development of the incidence of signs/symptoms, to prevent the development of the incidence of signs/symptoms, and/or effect prophylaxis of the incidence of signs/symptoms. The “effective amount” may vary depending on the disease and its severity and the age, weight, medical history, susceptibility, and pre-existing conditions, of the patient to be treated.

The terms “a” and “an,” when used to modify the ingredient of a composition, such as, active agent, buffering agent, and osmolyte, do not denote a limitation of quantity, but rather denote the presence of at least one of the referenced items. The term “or” or “and/or” is used as a function word to indicate that two words or expressions are to be taken together or individually. The terms “comprising,” “having,” “including,” and “containing” are to be construed as open-ended terms (i.e., meaning “including, but not limited to”). The endpoints of all ranges directed to the same component or property are inclusive and independently combinable.

Throughout the present specification, the terms “about” and/or “approximately” may be used in conjunction with numerical values and/or ranges. The term “about” is understood to mean those values near to a recited value. For example, “about 1200 [units]” may mean within ±10% of 1200, within ±10%, ±9%, ±8%, ±7%, ±6%, ±5%, +4%, ±3%, ±2%, ±1%, less than ±1%, or any other value or range of values therein. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term, “excipient,” as used herein, refers to any substance that may be formulated with defibrotide and may be included for the purpose of enhancement of the defibrotide in the final dosage form, such as facilitating its bioavailability, reducing viscosity and/or osmolality, enhancing solubility of the composition or to enhance long-term stability. Excipients can also be useful in the manufacturing process, to aid in the handling of the active substance. The selection of appropriate excipients also depends upon the route of administration and the dosage form, as well as the active ingredient and other factors. Accordingly, defibrotide may be combined with any excipient(s) known in the art that allows tailoring its performance during manufacturing or administration as well as its in vitro and in vivo performance. Many of these excipients may be utilized to tailor the pharmacokinetic profiles of defibrotide formulations.

The term, “formulation,” as used herein, refers to compositions for therapeutic use, including, for example, a stable and pharmaceutically acceptable preparation of a pharmaceutical composition or formulation disclosed herein.

The term, “high concentration formulation” or “high concentration liquid formulation” or “HCLF” as used herein, refers to those formulations where the concentration of the nucleic acid is about 80 mg/mL or higher; or about 85 mg/mL or higher. In some aspects, the defibrotide is a high concentration, low viscosity defibrotide formulation. In some embodiments, the high concentration, low viscosity defibrotide formulation is one disclosed in U.S. application Ser. No. 16/105,319 filed Aug. 3, 2018 the contents of which are incorporated herein for all purposes.

The term, “high concentration defibrotide formulations” as used herein, refers to those formulations where the defibrotide concentration is about 80 mg/mL or higher, or about 85 mg/mL or higher.

The term, “low dose” as used herein, refers to those formulations where the defibrotide administered at about 25 mg/kg/d or lower, 12.5 mg/kg/d or lower, or about 5 mg/kg/d or lower.

The term “NaCl control”, “NaCl group”, “NaCl control group”, or “NaCl treatment”, as used herein, refers to a saline group, saline treated group or vehicle control group. Saline is 0.9% w/v of NaCl.

The term, “pharmacokinetic” or “PK” as used herein, refers to in vivo movement of an individual agent in the body, including the plasma concentration time profiles and kinetic parameters like the maximum concentration (Cmax), area under the curve (AUC), and time to maximum concentration of said agent (Tmax).

The phrase “pharmaceutically acceptable” or “acceptable”, as used in connection with compositions of the disclosure, refers to molecular entities and other ingredients of such compositions that are physiologically tolerable and do not typically produce untoward reactions when administered to an animal and/or human. Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

As used herein, the term “parenteral” refers to any non-oral means of administration. It includes intravenous (i.v. or IV) infusion, IV bolus injection, subcutaneous (s.c. or SC) and intramuscular (i.m. or IM) injection. In some embodiments, defibrotide is administered intraveneously. In some embodiments, defibrotide is administered subcutaneously. Defibrotide which may be administered subcutaneously may require less frequent dosing than defibrotide products currently on the market are provided. In some embodiments, a device used to administer defibrotide is one disclosed in PCT Application No. PCT/US2019/064901 filed Dec. 6, 2019 the contents of which are incorporated herein for all purposes. In some embodiments, a device used to administer defibrotide is one disclosed in U.S. Application No. 62/802,099 filed Feb. 6, 2019 or U.S. Application No. 62/983,023 filed Feb. 28, 2020, the contents of both which are incorporated herein for all purposes.

As used herein, “prophylaxis” refers to action taken to prevent disease, especially by specified means or against a specified disease.

### Defibrotide and its Mechanisms of Action

Defibrotide (CAS number 83712-60-1) is a substance derived from materials of natural origin. Defibrotide, a nucleic acid salt, is a highly complex mixture of random sequences, predominantly single-stranded polydeoxyribonucleotides (predominantly single stranded and approximately 10% double stranded) derived from animal mucosal DNA Defibrotide has pleotropic biologic effect leading to the stabilization of endothelial cells, and in particular, defibrotide has protective effects on vascular endothelial cells, particularly those of small vessels and has antithrombotic, anti-inflammatory and anti-ischemic properties.

Defibrotide has a diverse size range and is known to have a mean molecular weight (MW) between 13 and 20 kDa. Defibrotide can be obtained according to U.S. Pat. Nos. 4,985,552 and 5,223,609 and/or presents the physical/chemical characteristics described in the same U.S. Pat. Nos. 4,985,552 and 5,223,609, each of which is incorporated herein by reference. Synthetic defibrotide, presented as phosphodiester oligonucleotides that mimic the therapeutic action of defibrotide are described in US20110092576 which is incorporated herein by reference in its entirety.

Defibrotide has numerous therapeutic applications, including use as an anti-thrombotic agent (U.S. Pat. No. 3,829,567), treatment of peripheral arteriopathies (U.S. Pat. No. 5,081,109), treatment of acute renal insufficiency (U.S. Pat. No. 4,694,134), treatment of acute myocardial ischemia (U.S. Pat. No. 4,693,995), topical treatments (U.S. Pat. No. 5,116,617) among other uses described in U.S. Pat. Nos. 3,770,720, 3,899,481, 4,938,873, 4,985,552, 5,223,609, 5,646,127, 5,646,268, 6,046,172, 6,699,985, 6,767,554, 7,338,777, 8,551,967, 8,771,663, 9,902,952 and 10,393,731; US Patent Publication Nos. 20080194506; 20090131362; 20110092576; 20130231470; 20140005256, U.S. Patent application Ser. Nos. 14/019,674; 14/323,918; 14/408,272; and International applications WO 2013/190582 and PCT/EP2015/077355; all of the preceding patents are incorporated herein by reference in their entireties. More recently, defibrotide has been used for the treatment and prevention of sinusoidal obstruction syndrome/veno-occlusive disease (EU clinical trial EudraCT:2004-000592-33, US clinical trial 2005-01 (ClinicalTrials.gov identifier: NCT00358501). Defibrotide is currently sold under the name Defitelio® as a single vial for injection (commercially available from Gentium S.r.L., Villa Guardia, Italy; see package insert available at dailymed.nlm.nih.gov/dailymed/search.cfm? labeltype=all&query=defibrotide). Defitelio® is prepared as an intravenous infusion by a dilution in 5% Dextrose Injection, USP or 0.9% Sodium Chloride Injection, USP. Intravenous preparation is used within 4 hours if stored at room temperature or within 24 hours if stored under refrigeration. It is administered for a total of 8 hours over 4 intravenous infusions.

In some embodiments, defibrotide is administered as a “high concentration formulation” (HCLF). In some embodiments the concentration of the nucleic acid in the HCLF is about 80 mg/mL or higher; or about 85 mg/mL or higher. In some aspects, the defibrotide is a high concentration, low viscosity defibrotide formulation. In some embodiments, the high concentration, low viscosity defibrotide formulation is one disclosed in U.S. application Ser. No. 16/105,319 filed Aug. 3, 2018 the contents of which are incorporated herein for all purposes.

In another embodiment, low dose defibrotide is administered prophylactically to a subject in need thereof before the onset of sepsis. In some embodiments, low dose defibrotide is about 25 mg/kg/d or lower, 12.5 mg/kg/d or lower, or about 5 mg/kg/d or lower.

### Defibrotide Administration

In some embodiments, defibrotide may be administered to prevent, ameliorate, delay, or treat sepsis in a patient who is suffering from an infection. In some embodiments, defibrotide is administered to prevent, ameliorate, delay, or treat dysregulated inflammatory responses in a patient who is suffering from sepsis. In some embodiments, defibrotide is administered to prevent, ameliorate, delay, or treat sepsis or other dysregulated inflammatory responses in a patient who is suffering from pancreatitis. In some embodiments, defibrotide is administered to prevent, ameliorate, delay, or treat sepsis or other dysregulated inflammatory responses in a patient who is suffering from pneumonia. In some embodiments, defibrotide is administered to prevent, ameliorate, delay, or treat sepsis in a patient who is suffering from an infection caused by one or more viruses. In some embodiments, defibrotide is administered to prevent, ameliorate, delay, or treat sepsis or other dysregulated inflammatory responses in a patient who is suffering from an infection caused by one or more coronaviruses.

In some embodiments, defibrotide is administered to prevent, ameliorate, delay, or treat sepsis or other dysregulated inflammatory responses in a patient who is suffering from lung trauma. In some embodiments, defibrotide is administered to prevent, ameliorate, delay, or treat sepsis or other dysregulated inflammatory responses in a patient who is suffering from lung injury.

In some embodiments, defibrotide may be administered to a patient diagnosed as having been infected with SARS-CoV-2. In some embodiments, defibrotide may be administered to a patient displaying one or more symptoms of SARS-CoV-2 infection. In some embodiments, defibrotide may be administered to a patient suspected of being infected with SARS-CoV-2. In some embodiments, defibrotide may be administered to a patient diagnosed with COVID-19. In some embodiments, defibrotide may be administered to a patient displaying one or more symptoms of COVID-19. In some embodiments, defibrotide may be administered to an asymptomatic patient infected with SARS-CoV-2.

In some embodiments, defibrotide may be administered to a patient diagnosed with, or suspected of having, acute respiratory distress syndrome.

In certain embodiments, defibrotide is administered prophylactically. In some embodiments, one or more administrations of defibrotide are administered prophylactically to a patient determined to be at high-risk of developing sepsis, other dysregulated inflammatory responses, or a related disorder. In some embodiments, the one or more administrations of the defibrotide begins before the patient develops sepsis, other dysregulated inflammatory responses, or a related disorder. In some embodiments, the one or more administrations of the defibrotide begins before the patient starts showing symptoms of sepsis, other dysregulated inflammatory responses, or a related disorder. In some embodiments, the one or more administrations of the defibrotide begins after the patient shows an altered level of a biomarker associated with the development of sepsis, other dysregulated inflammatory responses, or a related disorder. In some embodiments, prophylactic administration of defibrotide prevents the development of sepsis, other dysregulated inflammatory responses, or a related disorder. In some embodiments, prophylactic administration of defibrotide delays the development of sepsis, other dysregulated inflammatory responses, or a related disorder. In some embodiments, prophylactic administration of defibrotide delays or ameliorates the development of one or more symptoms of sepsis, other dysregulated inflammatory responses, or a related disorder.

In some embodiments, the one or more defibrotide treatments may begin before the patient is diagnosed with sepsis, other dysregulated inflammatory responses, or a related disorder. In some embodiments, the one or more defibrotide treatments may begin on the same day as the patient was diagnosed with sepsis, other dysregulated inflammatory responses, or a related disorder or, for a variety of reason which are readily apparent to a skilled artisan, they may begin on a day after the patient was diagnosed with sepsis, other dysregulated inflammatory responses, or a related disorder. For example, the defibrotide treatments may begin on days −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 after the patient was diagnosed sepsis, or a related disorder. Thus, in some embodiments, the one or more administrations of the defibrotide begins on 3, 2, or 1 days before the patient diagnosed as having sepsis, other dysregulated inflammatory responses, or a related disorder. In some embodiments, the one or more administrations of the defibrotide begins on the same day that the patient diagnosed as having sepsis, other dysregulated inflammatory responses, or a related disorder (i.e., day 0). In other embodiment, the one or more administrations of the defibrotide begins on 1, 2, 3, 4, 5, 6, or 7 days after the patient diagnosed as having sepsis, other dysregulated inflammatory responses, or a related disorder. In some embodiments, the one or more administrations of defibrotide begin 1 day after the patient was diagnosed as having sepsis, other dysregulated inflammatory responses, or a related disorder.

The timing of the administration of the defibrotide may depend on the particular patient (e.g. whether the patient is at high-risk of developing sepsis, other dysregulated inflammatory responses, or a related disorder) and any additional therapies to be administered or co-administered.

The defibrotide may be administered as often and as for long as required. In some embodiments, the defibrotide is administered 1-120 times. In some embodiments, the defibrotide is administered for about one day, about two days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, or about 30 days or more. The defibrotide may be administered daily, weekly, or monthly. In some embodiments, the defibrotide is administered every day for about one week, about two weeks, about three weeks, or about four weeks.

As a skilled artisan will appreciate, a defibrotide treatment period may vary on a patient-by-patient basis. In some embodiments, a defibrotide treatment period may vary depending on the assessed likelihood of sepsis. In some embodiments, a defibrotide treatment period is determined by monitoring signs and symptoms of sepsis or consequences thereof. For example, if the signs and symptoms of sepsis or consequences thereof are still present after an initial treatment period, defibrotide treatment is continued until resolution of sepsis.

In some embodiments, a treatment period lasts from about 1 day to about 1 year, for example about 1 day, 2 days, 3 days, 4 days, 5 days, 6 days, 7 days, 8 days, 9 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 6 weeks, 2 months, 3 months, 4 months, 5 months, 6 months, 7 months, 8 months, 9 months, 10 months, 11 months, or 12 months, or more, including all values and ranges in between thereof. In some embodiments, a treatment period lasts 1 week.

The defibrotide dosing may be determined by a variety of factors that will be readily apparent to a skilled artisan. In some embodiments, a dose is based on patient's baseline body weight. In some embodiments, defibrotide is administered in an amount of about 1 to about 100 mg/kg of body weight per day. For example defibrotide is administered in an amount of about 1 mg/kg, about 1.25 mg/kg, about 1.50 mg/kg, about 1.75 mg/kg, about 2 mg/kg, about 2.25 mg/kg, about 2.50 mg/kg, about 2.75 mg/kg, about 3 mg/kg, about 3.25 mg/kg, about 3.50 mg/kg, about 3.75 mg/kg, about 4.25 mg/kg, about 4.50 mg/kg, about 4.75 mg/kg, about 5 mg/kg, about 5.25 mg/kg, about 5.50 mg/kg, about 5.75 mg/kg, about 6 mg/kg, about 6.25 mg/kg, about 6.50 mg/kg, about 6.75 mg/kg, about 7 mg/kg, about 7.25 mg/kg, about 7.50 mg/kg, about 7.75 mg/kg, about 8 mg/kg, about 8.25 mg/kg, about 8.50 mg/kg, about 8.75 mg/kg, about 9 mg/kg, about 9.25 mg/kg, about 9.50 mg/kg, about 9.75 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, or about 100 mg/kg of body weight per day, including all ranges therebetween. In some embodiments, defibrotide is administered in an amount of about 25 mg per kilogram of body weight per day. In some embodiments, doses based on the patient's body weight were rounded to the nearest 10 mg for patients over 35 kg. In some embodiments, doses based on the patient's body weight were rounded to the nearest 5 mg for patients under 35 kg. In some embodiments, the dose is 25 mg/kg/day. In some embodiments, the dose is 25 mg/kg/dose. In some embodiments, the dose is 2.5 mg/kg/dose. In some embodiments, the dose is 6.25 mg/kg/dose. In some embodiments, defibrotide is administered as a high concentration low viscosity formulation, as described in WO 2019/028340 the contents of which are incorporated by reference in their entirety for all purposes.

The defibrotide may be administered as a single daily dose or in multiple doses per day. In some embodiments, defibrotide is administered once a day. In some embodiments, defibrotide is administered in multiple doses per day. For example, defibrotide may be administered in 2, 3, 4, 5, 6, 7, 8, 9, or in 10 doses per day. In some embodiments, defibrotide is administered in four doses per day. In some embodiments, defibrotide is administered in four doses per day every 6 hours. In some embodiments, defibrotide is administered by continuous infusion.

In some embodiments, when defibrotide is administered in multiple doses per day, the different doses of defibrotide are administered from about 30 minutes to about 12 hours apart. In some embodiments, defibrotide is administered about every 30 minutes, about every 40 minutes, about every 50 minutes, about every 60 minutes, about every 70 minutes, about every 80 minutes, about every 90 minutes, about every 2 hours, about every 3 hours, about every 4 hours, about every 5 hours, about every 6 hours, about every 7 hours, about every 8 hours, about every 9 hours, about every 10 hours, about every 11 hours, or about every 12 hours. In some embodiments, when defibrotide is administered in multiple doses per day, the different doses of defibrotide are administered about 6 hours apart.

The defibrotide may be administered daily, weekly, or monthly. In some embodiments, the defibrotide is administered every day for about one week, about two weeks, about three weeks, or about four weeks, or more. In some embodiments, defibrotide administration occurs on consecutive days. In some embodiments, defibrotide administration occurs on discontinuous days.

In some embodiments, defibrotide administration occurs on consecutive days. In some embodiments, defibrotide administration occurs on discontinuous days.

In some embodiments, the one or more administrations of defibrotide are administered to treat symptoms of sepsis or other dysregulated inflammatory responses. In some embodiments, the defibrotide is administered until symptoms improve. In some embodiments, the defibrotide is administered until symptoms are eradicated. In some embodiments, the defibrotide is administered until sepsis, other dysregulated inflammatory responses, or related disorder is cured.

As a skilled artisan will appreciate, the defibrotide treatment period may vary on a patient-by-patient basis. In some embodiments, the defibrotide treatment period is determined by monitoring signs and symptoms of sepsis, other dysregulated inflammatory responses, or a related disorder. For example, if the signs and symptoms of sepsis, other dysregulated inflammatory responses, or a related disorder are still present after an initial treatment period, defibrotide treatment is continued until resolution of sepsis, other dysregulated inflammatory responses, or a related disorder.

Any combination of the foregoing embodiments may be used in treating the patient with defibrotide. Accordingly, in some embodiments, a patient is administered defibrotide intravenously in an amount of about 2.5 mg per kilogram of body weight about every 6 hours. Accordingly, in some embodiments, a patient is administered defibrotide intravenously in an amount of about 6.25 mg per kilogram of body weight about every 6 hours.

The defibrotide may be administered by any suitable route, including without limitation parenteral (e.g., intravenous, subcutaneous, intrasternal, intramuscular, or infusion techniques), oral, sublingual, buccal, intranasal, pulmonary, topical, transdermal, intradermal, mucosal, ocular, otic, rectal, vaginal, intragastric, intrasynovial, and intra-articular routes. In some embodiments, defibrotide is administered intravenously. In some embodiments, defibrotide is administered via intravenous infusion. In some embodiments, defibrotide is administered by constant intravenous infusion over a 2-hour period. In some embodiments, the defibrotide is diluted prior to infusion. In some embodiments, the diluted defibrotide solution is administered using an infusion set equipped with a filter (e.g., a 0.2 micron in-line filter). In certain embodiments, the intravenous administration line (e.g., peripheral or central) is flushed immediately before and after administration (e.g., with 5% Dextrose Injection, USP or 0.9% Sodium Chloride Injection, USP).

In some embodiments, the defibrotide is administered subcutaneously. In some embodiments, the defibrotide is administered subcutaneously by means of a device that is commercially available such as, for example, the FREEDOM60® pump or similar (RMS™ Medical Products). In some embodiments, the defibrotide is administered subcutaneously using an automated injection device. Subcutaneous administration of a high concentration low viscosity defibrotide formulation via an automated injection device may offer significant reduction of the time for clinical administration and enable outpatient dosing of the product for as long as needed. The use of an automated injection device improves convenience and allows faster administration by health-care professionals (HCP), caregivers, or even self-administration by the patients.

In some embodiments, the route of administration affects the efficacy and/or longevity of the formulations of the present disclosure. In some embodiments, subcutaneous, intramuscular and/or intraperitoneal administration is associated with an extended systemic half-life compared to the same formulation administered intravenously. In some embodiments, subcutaneous administration of the formulation provides lower peak-to-trough ratios of plasma concentrations compared to the same formulation administered intravenously. In some embodiments, subcutaneous administration provides improved efficacy and/or improves the safety profile of the formulation compared to the same formulation administrated intravenously.

Devices for subcutaneous administration may be prefilled, with for example a predefined adult or pediatric dose, or may be used to administer a weight-based dose specific for individual patients. In some embodiments, the patient determines the dose and administers it. In some specific embodiments, formulations of the disclosure are administered subcutaneously in less than about two hours, less than about one hour, or less than about 30 minutes. In some specific embodiments, formulations of the disclosure are delivered subcutaneously over about 5 minutes to about 1 hour, about 10 minutes to about 1 hour or about 15 minutes to about 45 minutes.

In some embodiments, subcutaneous administration of the low-viscosity formulations of the present disclosure allows for less-frequent administration and/or lower doses. In some embodiments, subcutaneous administration of the low-viscosity formulation of the present disclosure allows for reduced administration volume.

In accordance with some embodiments of the present disclosure, a patient is from about 0 years of age to about 16 years of age, including all ranges and subranges therein. For example, a patient is from about 0 months, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, to about 16 years of age. In some embodiments, a patient is from about 0 months to about 23 months of age. In some embodiments, a patient is from about 2 years to about 11 years of age. In some embodiments, a patient is from about 12 years to about 16 years of age. In accordance with some embodiments of the present disclosure, a patient may be a pediatric patient or adult. A pediatric patient is from about 0 years of age to about 16 years of age, including all ranges and subranges therein. For example, a pediatric patient is from about 0 months, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, to about 16 years of age. In some embodiments, a patient is from about 0 months to about 23 months of age. In some embodiments, a patient is from about 2 years to about 11 years of age. In some embodiments, a patient is from about 12 years to about 16 years of age.

In some embodiments, a patient is an adult patient. An adult patient is older than 16 years of age. In some embodiments, the adult patient is 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, or 105 years of age. In some embodiments, an adult patient is between 16 and 30 years of age, including all values and ranges in between. In some embodiments, an adult patient is between 31 and 40 years of age, including all values and ranges in between. In some embodiments, an adult patient is between 41 and 50 years of age, including all values and ranges in between. In some embodiments, an adult patient is between 51 and 60 years of age, including all values and ranges in between. In some embodiments, an adult patient is between 61 and 70 years of age, including all values and ranges in between. In some embodiments, an adult patient is between 71 and 80 years of age, including all values and ranges in between. In some embodiments, an adult patient is between 81 and 90 years of age, including all values and ranges in between. In some embodiments, an adult patient is between 91 and 100 years of age, including all values and ranges in between. In some embodiments, a patient older than 65 years old is considered to be at higher risk of developing sepsis, other inflammatory lung disorders, or related conditions.

In some embodiments, the patient who is considered at high-risk of developing sepsis, other dysregulated inflammatory responses, or related conditions is selected from a group consisting of an adult 65 or older, an infant younger than one, a person with a chronic condition, a person with a weakened immune system, and a pregnant woman. The chronic conditions include, but are not limited to, diabetes, cancer, lung disease, kidney disease and liver disease.

The following examples further illustrate various embodiments of the present disclosure, but should not be construed in any way as limiting its scope.

### EXAMPLES

### Example 1—Efficacy of Defibrotide Prophylaxis for the Treatment of Sepsis Study: to evaluate the prophylactic effect of defibrotide for the treatment of sepsis

### Materials and Methods

Pathogen-free male and female C57BL/6JRj mice were purchased from JANVIER lab (Le Genest-Saint-Isle, France) and have been used at an age of 6-8 weeks. Experiments were initiated after an acclimatization period of one week. Mice were housed in a 12-hour light/dark cycle.

Adult males and females C57BL/6JRj were placed separately in 5 groups: one control group with 4 mice (receiving NaCl), and four groups containing 8 mice per group challenged with LPS. To determine the doses inducing 50% lethality (LD50), four LPS doses from E. coli (serotype 0111:B4, from Sigma) were injected intravenously in the caudal tail vein: 5 mg/kg, 10 mg/kg, 20 mg/kg or 30 mg/kg dissolved in 0.9% NaCl (Ecotainer®, B Braun, Germany). To limit pain caused by LPS action, buprenorphine (Temgesic, 0.3 mg/mL) was injected subcutaneously at 0.1 mg/kg in all mice, 30 minutes before LPS intravenous injection in the caudal tail vein. LPS induced sepsis resulted in a strong inflammatory response in the first 48 hours, with a significant impact on the clinical features and finally causing death of the animal such as: loss of body weight, change in the body temperature, piloerection which is a typical feature of the fur in infection disease, less active, isolated from the group, difficulty to breath, change in the facial grimacing. Therefore, buprenorphine (Bupaq, 0.3 mg/10 mL) was added to the drinking water at 1 mg/kg for 48 hours. To improve intravenous injection in C57BL/6JRj mice a homemade illuminator system (Messer 2015) and a heating lamp were used.

The mice were followed for 4 days. Every day, the body temperature and weight were measured by an infrared thermometer (MDI907, Mediwear, Finland) and a scale respectively. The condition of the animals was assessed using an adapted version of Clinical Severity Score (CSS) (Shrum et al. 2014). Mice were euthanized if the cumulative score of the clinical features (fur aspect, activity, posture, respiratory, facial grimace, body weight loss and body temperature loss) was greater than 8 or when the body weight loss exceeded 20% together with changes in others parameters in the 48 hours. Mice were euthanized by intraperitoneal pentobarbital injection (Eskonarkon 150 mg/mL).

Defibrotide was obtained from Jazz pharmaceutical Company in vials of 200 mg in 2.5 mL, corresponding to 80 mg/mL. Defibrotide was diluted in pyrogen-free sterile 0.9% NaCl (Ecotainer®, B Braun, Germany) and injected thrice a day intraperitoneally. The injection was performed 2 hours before (prophylaxis treatment) or 2 hours after (therapeutic treatment) administration of LPS, followed by administrations every 8 hours at concentrations of 8.3 mg/kg, 26.7 mg/kg or 58.3 mg/kg per injection, corresponding to 25 mg/kg/day, 175 mg/kg/day and 350 mg/kg/day, respectively.

Total blood was collected from vena cava in tubes containing ethylenediaminetetraacetic acid-K2E (EDTA-K2 E, BD microtainer) as an anticoagulant. After centrifugation at 3000 rcf for 15 min at 4 °C, plasma was separated from blood cells and stored at −80 °C. Aspartate transaminase (ASAT), alanine transaminase (ALAT), creatinine, urea and lactate-dehydrogenase (LDH) were obtained via routine chemistry laboratory measurements in the Cobas 8000 (Roche, Switzerland)

Organs were harvested: spleen, lungs and left lobe of liver has been placed in cold tubes with beads. Then, 1mL of lysis buffer (1X PBS (pH7.4) 0.1%Triton X100 and 1% Protease inhibitor cocktail (P8340, Sigma-Aldrich) was added to each tube. Tissues were lysed for 1 min30 sec at the frequency 30 sec⁻¹ by using a Tissuelyser from Qiagen. Thereafter, the lysed tissue was centrifuged at 12 000 rcf for 10 min at 4 °C and the supernatant was store at −80 °C until further use. Mouse cytokines (TNF-α, IL-1β and IL-6), mouse myeloperoxidase (MPO), CXCL-1/KC; ICAM-1/CD54 (R&D systems, Abingdon, UK) were performed according to the manufacturer's protocol and as previously described (van der Meer et al. 2019).

Nucleosome levels were determined with an ELISA as previously described (Zeerleder et al. 2007; van der Meer et al. 2019). Briefly, ELISA plates were coated with monoclonal anti-histone H3 antibody (CLB/ANA-60) and the samples were added and incubate for 1 hour at room temperature. After washing, biotin-labelled F(ab′)2 fragments of monoclonal anti-nucleosome antibody (CLB/ANA-58) were added and incubated for another hour at room temperature. Binding of biotin-labelled antibodies was detected with streptavidin-horse radish peroxidase (HRP) using tetramethylbenzidine (TMB) as a substrate. The reaction was stopped with 2 M H₂SO4 and the absorbance was measured at 450 nm.

All statistical analyses and Figures were computed with GraphPad Prism software v 9.3 (GraphPad Software, La Jolla, CA, USA). All the experiments were performed with 8 animals per group, during five days for the survival study, then per time point of each experimental group, except for control mice without sepsis and defibrotide injection (n=4). For the survival rate, statistical significance was determined by using Mantel-Cox test. For all other experiments, statistical significance was determined by using the non-parametric Kruskal-Wallis test, followed by the Dunn's multiple comparison test. If the Dunn test was not significant and we could clearly see a difference between two groups, then Mann-Whitney test was used. All the groups treated were compared with untreated group for a same time point. Then, all the groups from a same treatment were compared at different time points. Data are presented as mean±standard deviation error of mean (SEM).

### Results

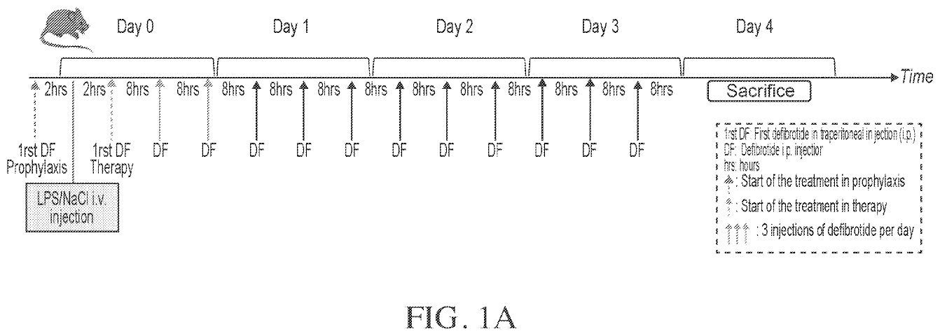

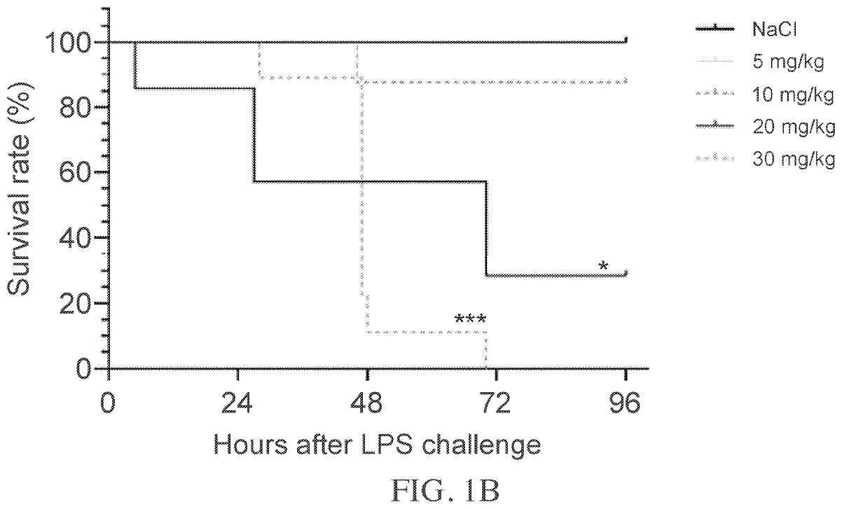

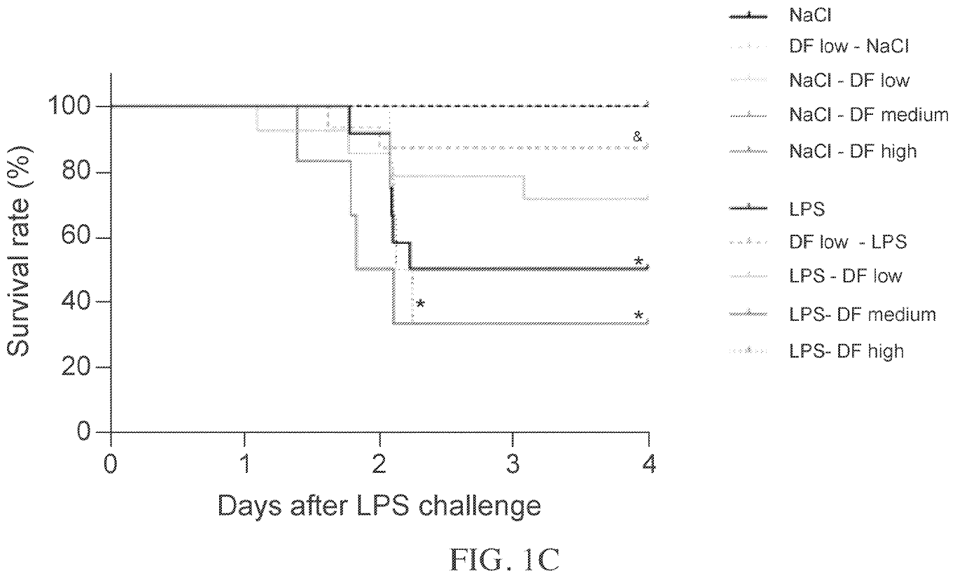

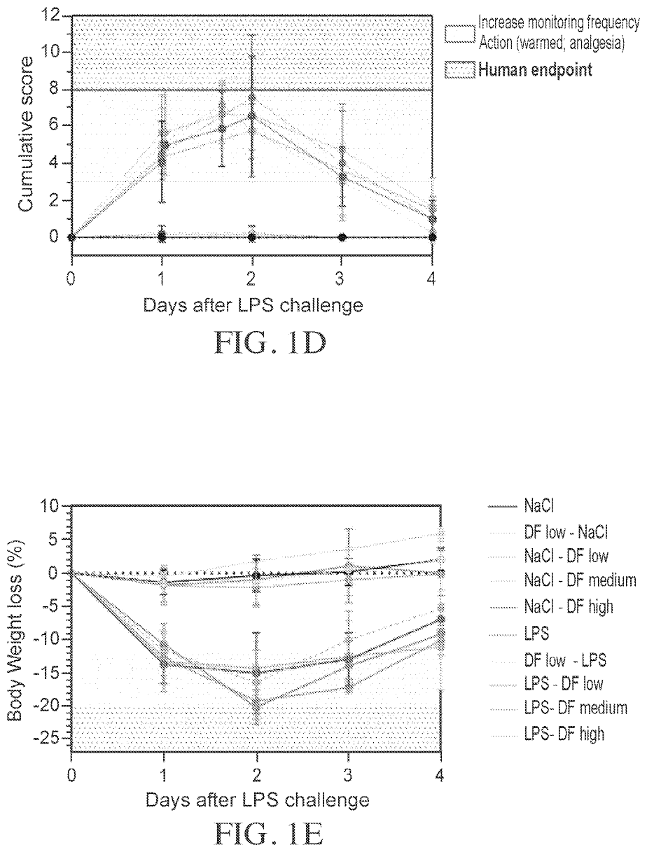

FIG. 1A illustrates the protocol for evaluating the impact of defibrotide on survival rate and clinical parameters in an endotoxemia mouse model. In a first step, different LPS concentrations were tested to identify the LPS dose leading to 50% mortality (LD50) (FIG. 1B). After LPS challenge, the mice were followed for 4 days (corresponding to 96 hours). With low doses of LPS (5 mg/kg and 10 mg/kg) the survival rate was 100%. In contrast, 48 hours after LPS challenge with 20 mg/kg or 30 mg/kg the survival rate significantly decreased to 50% and 11%, respectively (*p=0.0214 and ***p=0.0004 respectively).

Based on these data, for all the following experiments LPS dose of 20 mg/kg was used. In the control groups no lethality was observed from any dose of defibrotide administered with saline to mice (FIG. 1C). The lowest dose of defibrotide (25 mg/kg/d) administered 2 hours after LPS resulted in a slight improvement of the survival rate (71.4% compared to 50% without defibrotide, not significant p=0.33). Interestingly, the two higher defibrotide doses (175 mg/kg/d and 350 mg/kg/d) administered 2 hours after LPS significantly decreased the survival rate for both groups (*p=0.0183). Prophylactic administration of defibrotide at low dose 2 hours before LPS induced a significant improvement of survival (87.5%) as compared to mice with LPS-induced sepsis without defibrotide treatment (p=0.0472).

A cumulative score to identify the pain intensity and the change in clinical features of the mice, such as fur aspect, activity, posture, respiratory, facial grimacing, body weight loss and decrease in body temperature was applied to all mice studied. The score in mice in the control groups injected with saline or DF only, did not change and reached a maximal score of 1 point (FIG. 1D). Mice with sepsis receiving prophylactic treatment with DF showed a trend to have better scores as compared to mice not receiving DF (5.8±2.3 vs 6.5±3.3; p=0.85) 2 days after LPS challenge.

The body weight loss after 24 hours is an important clinical feature to confirm that sepsis was efficiently induced in our model. Mice did not lose more than 5% of body weight and occasionally they even gained body weight after NaCl injection with and without DF. In all mice receiving LPS, body weight decreased until 2 days after the LPS challenge, resulting in a loss of more than 15% and 20% bodyweight for low and medium/high doses of defibrotide, respectively (FIG. 1E). In the following days, the gain in body weight of mice correlated with recovery. The change intra and inter groups were not significant. However, animals with sepsis treated with defibrotide at low dose in prophylaxis recovered faster with a change of body weight loss −16.4±1.5 % at 48 hours after LPS challenge and −10.0±4.3 % at 72 hours after LPS challenge (score for this clinical feature is at 3 and 1 respectively).

In summary, the data demonstrate that prophylactic defibrotide administration improves survival in endotoxemia.

### Effect of Defibrotide on Organ Function

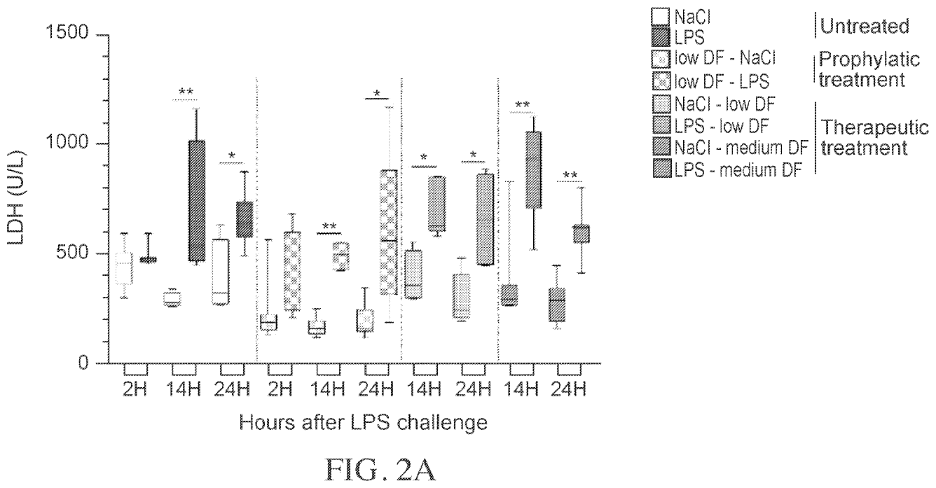

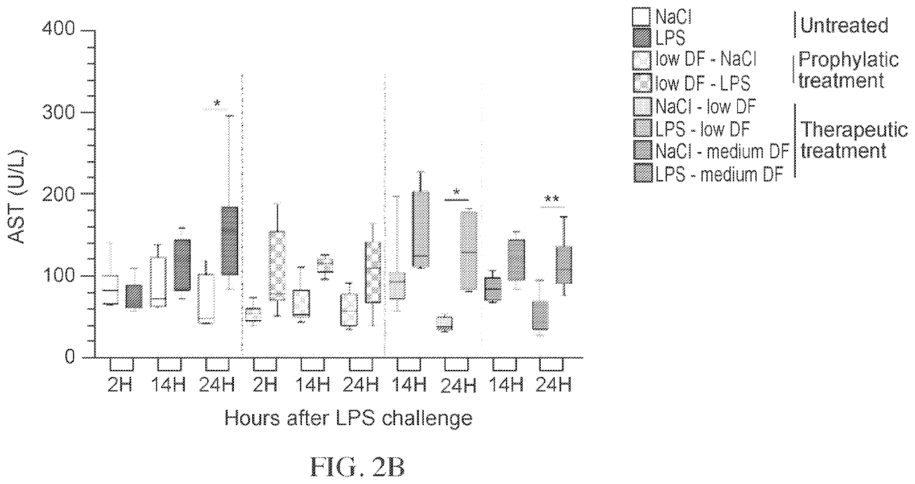

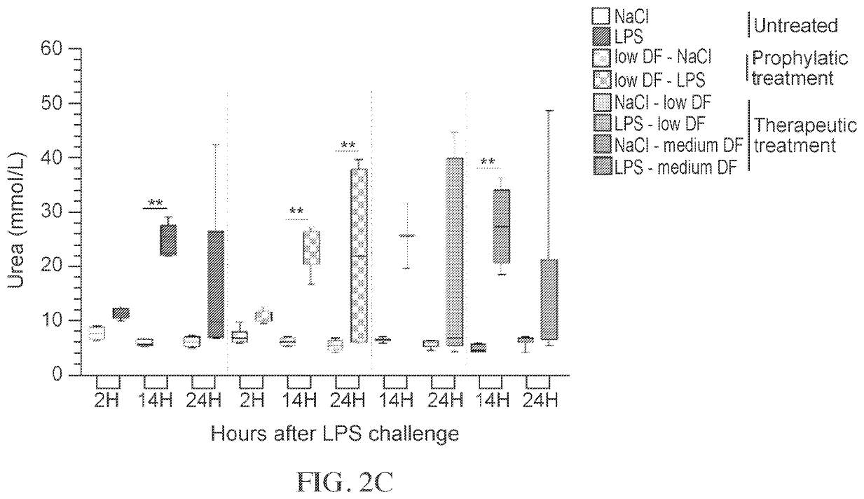

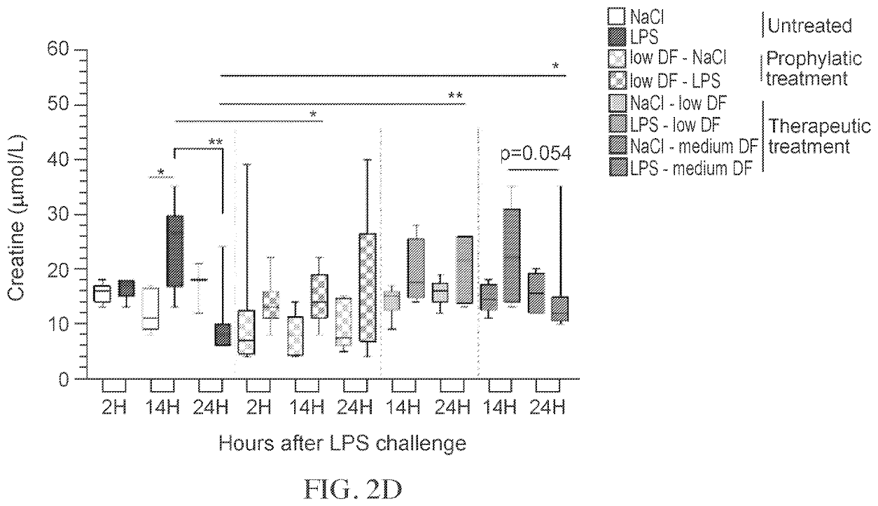

LPS induced significant increase of LDH as a reflection of systemic organ injury at 14 hours and 24 hours after injection in all LPS groups as compared to NaCl control groups treated or not treated with defibrotide (FIG. 2A). Defibrotide had no effect on systemic LDH levels.

In a next step, the effect of defibrotide in endotoxemia on the liver was studied. Systemic levels of AST and ALT were measured in the mice. Twenty-four hours after LPS administration, AST levels significantly increased in all mice irrespective whether treated or not treated with defibrotide (FIG. 2B). Nevertheless, in this sepsis model LPS induced no significant change of ALT levels in blood with or without treatment (data not shown). These results suggested that sepsis induced by LPS intravenous injection induces liver injury, and defibrotide treatment does not prevent or attenuate liver injury.

To assess kidney function systemic urea and creatinine levels were measured in mice. At 14 hours, LPS increased urea levels in all groups untreated and treated with defibrotide compared to NaCl control groups (**p<0.01) (FIG. 2C). Neither prophylactic nor therapeutic defibrotide treatments had an effect on urea levels. Creatinine levels significantly increased 14 hours after LPS challenge as compared with NaCl group (*p=0.045), and decreased (**p=0.0033) significantly 24 hours after endotoxemia induced (FIG. 2D). Defibrotide had an impact on creatinine levels. At 14 hours, prophylactic defibrotide significantly decreased creatinine levels in LPS group compared to LPS group receiving no DF (*p=0.046), and the levels remained stable at 24 hours. The therapeutic treatment had no impact on creatinine levels at 14 hours after sepsis-induced, nevertheless at 24 hours creatinine levels increased significantly compared to the LPS group untreated (**p=0.0082 with low dose, *p=0.015 with medium dose) . In prophylaxis treatment, defibrotide reduces creatinine levels whereas as therapeutic treatment defibrotide increases it.

### The Effect of Defibrotide on the Inflammatory Response in the Spleen, Lung, Liver and Systemically

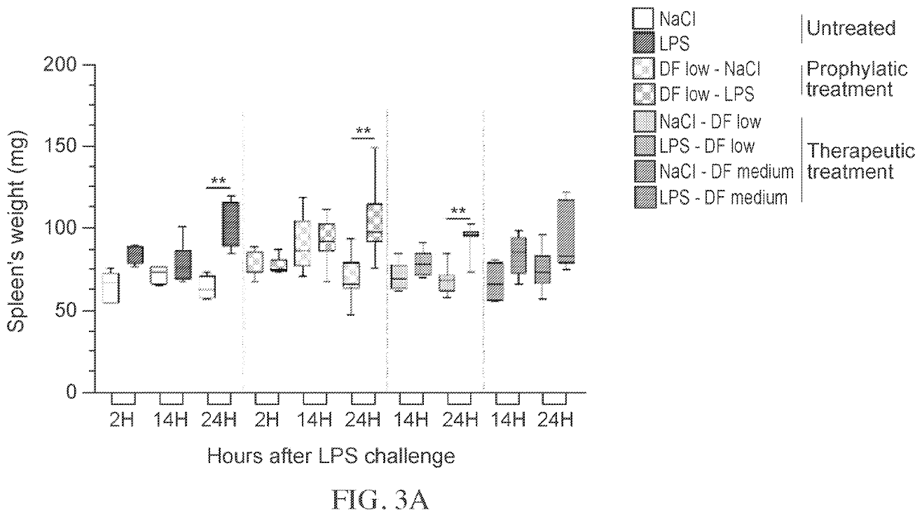

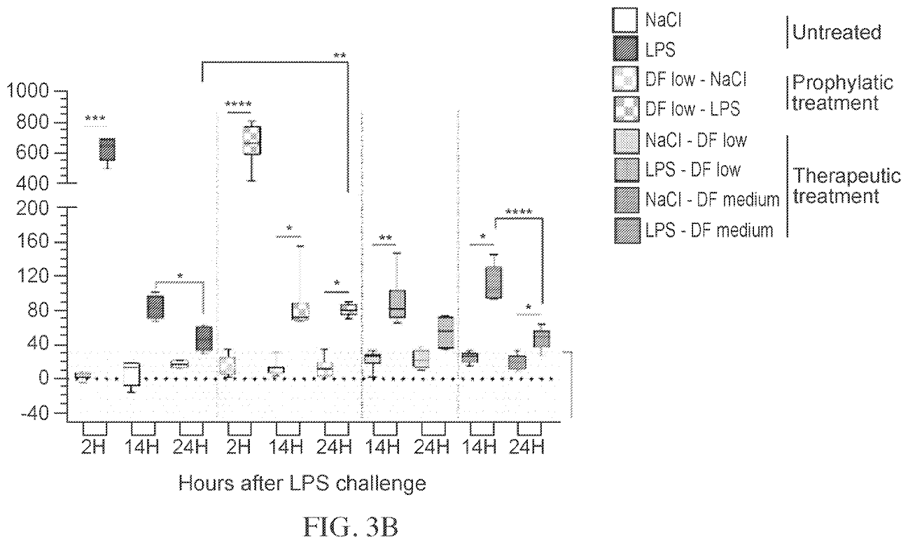

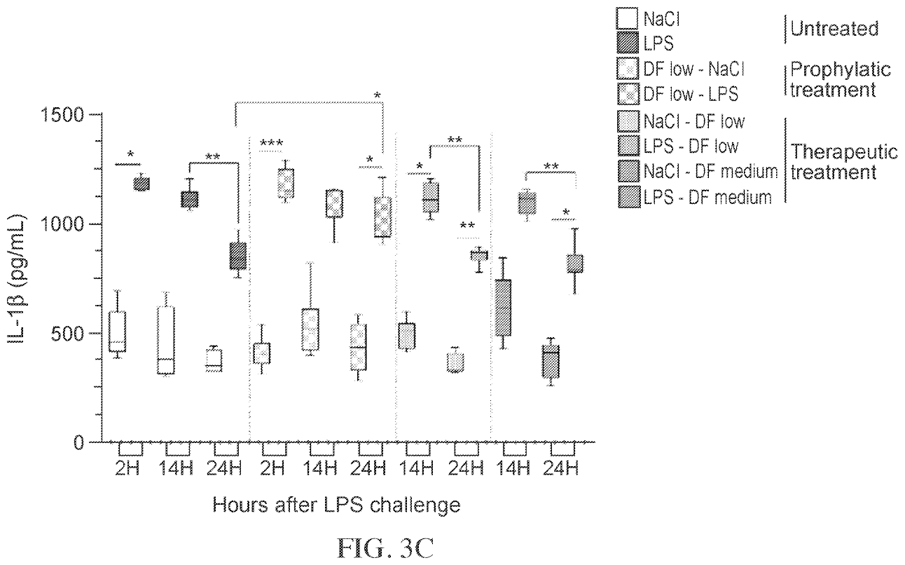

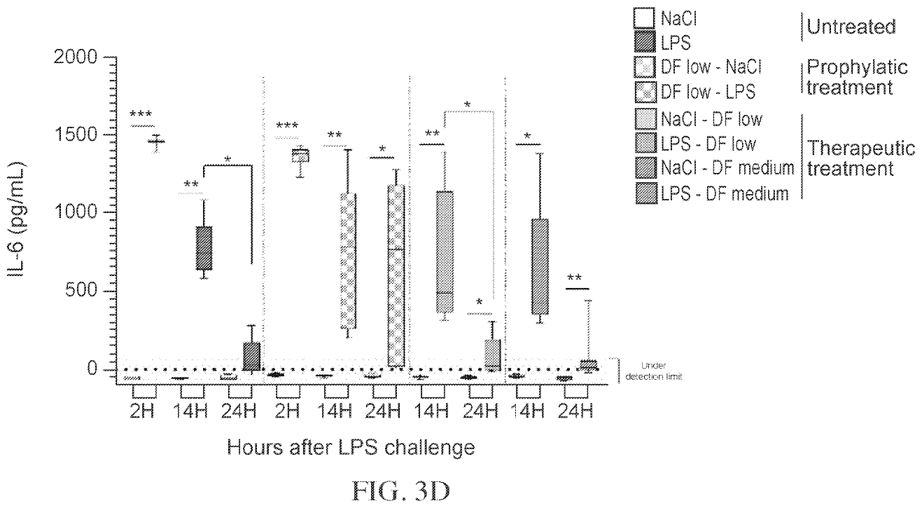

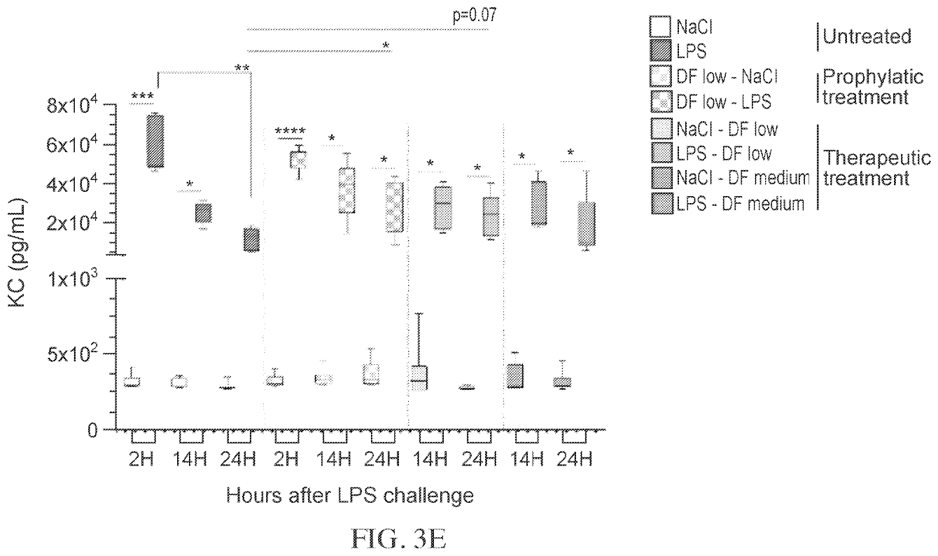

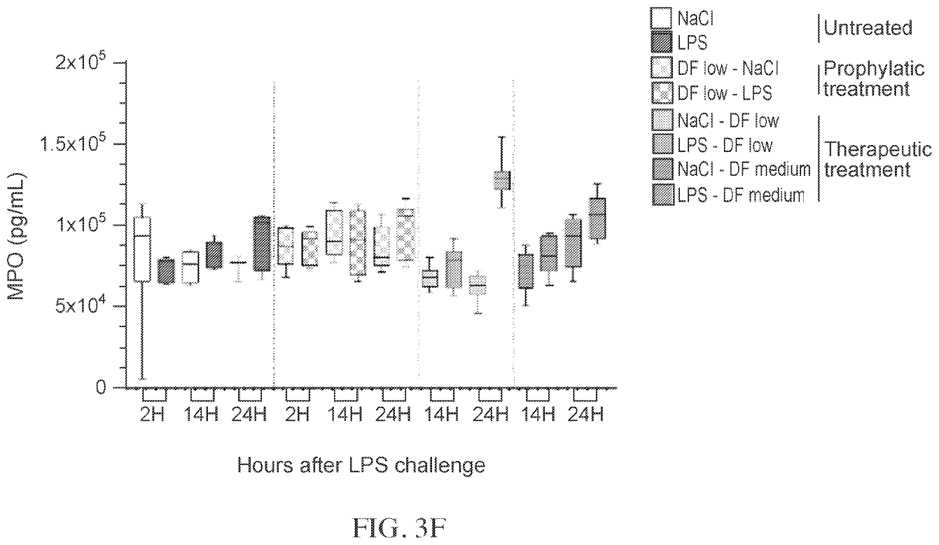

In a next step, host immune response in the spleen was measured. First, the spleen weight of the mice was measured (FIG. 3A). In all mice in the LPS group irrespective of whether treated or not with defibrotide spleen's weight significantly increased 24 hours after LPS administration compared to all control groups, but DF did not impact spleen weight in LPS treated mice.

Then, the inflammatory cytokines TNF-α, IL-1β and IL-6 were analyzed. LPS induced a significant production of TNF-a (624.6 ±36.5 pg/mL) compared to NaCl group (4.4 ±2.0 pg/mL) 2 hours after administration, with a subsequent significant decrease at 14 hours and 24 hours after sepsis induction (83.5±5.5 pg/mL vs 46.6±6.3 pg/mL respectively, *p=0.022). Interestingly, although prophylactic administration of defibrotide had no effect on LPS-induced TNF-α production 2 and 14 hours after LPS challenge, TNF-α was significantly higher at 24 hours (**p=0.006) . Therapeutic defibrotide treatment did not show any effect on the TNF-α production. A similar course was observed for IL-1β (FIG. 3C). Two hours after LPS challenge LPS induced a significant increase in IL-1β, as compared to the NaCl control, followed by a decrease in IL-1β levels 14 and 24 hours after LPS challenge. After prophylactic DF administration, IL-1β release increased 2 hours after LPS-induced sepsis similar to the untreated LPS group. As with TNF-α, IL-1β production was significantly higher at 24 hours in the defibrotide group than LPS only (*p=0.033). IL-6 production significantly increased 2 hours after LPS administration followed by a significant gradual decrease 14 and 24 hours after LPS challenge in the non-treated groups (FIG. 2D). DF had no effect on the IL-6 levels in endotoxemia.

LPS induced a significant increase in KC production 2 hours after LPS challenge, followed by a significant gradual decrease in KC production 14 and 24 hours after LPS challenge (FIG. 3E, **p=0.001). At 24 hours, prophylactic administration of DF induced an increase in KC production as compared to the untreated LPS group (*p=0.0295) and a similar trend was observed with therapeutic DF at low dose (p=0.07). Since KC is important in neutrophil recruitment, MPO levels were analyzed in the mice. MPO levels did not significantly increase after LPS challenge and MPO was comparable in DF-treated and non-treated mice, respectively, indicating that there was no significant effect of defibrotide on neutrophil recruitment (FIG. 3F).

Together, these data on TNF-α, IL-1β and KC in the spleen suggest that prophylactic treatment with DF induces a prolonged inflammatory response.

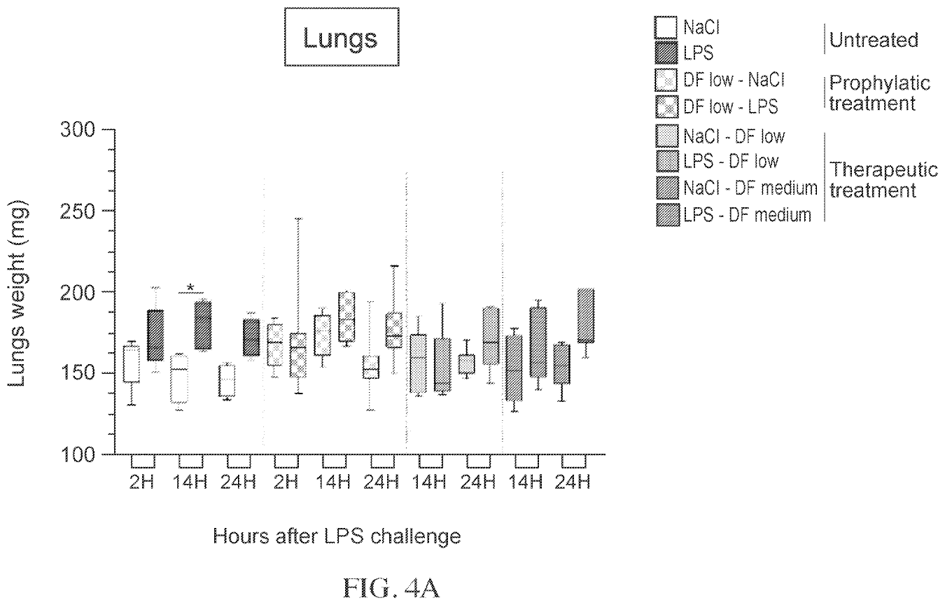

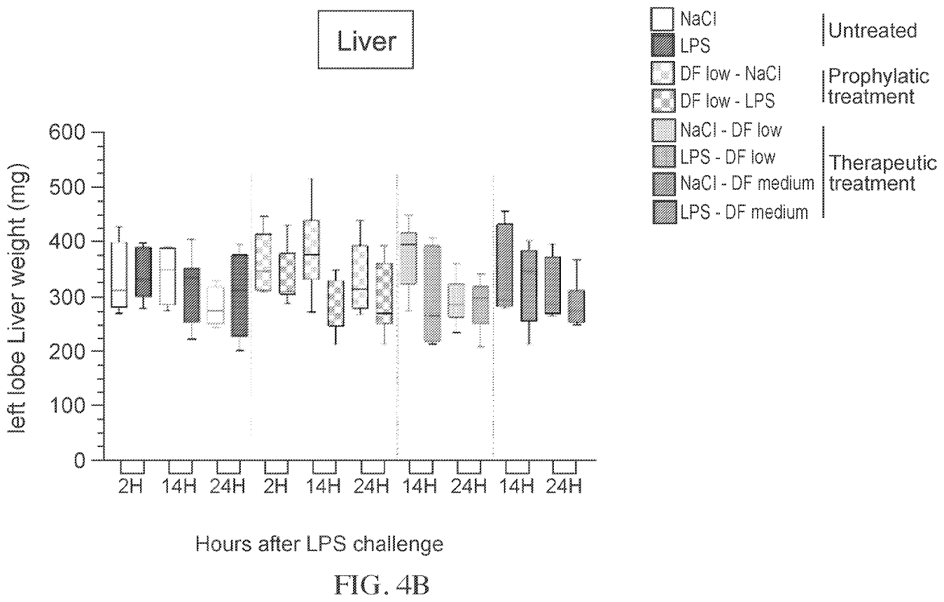

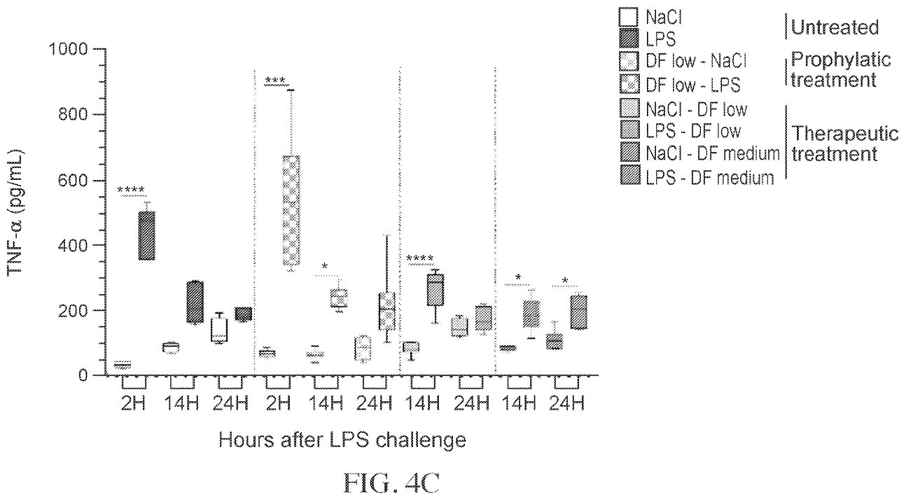

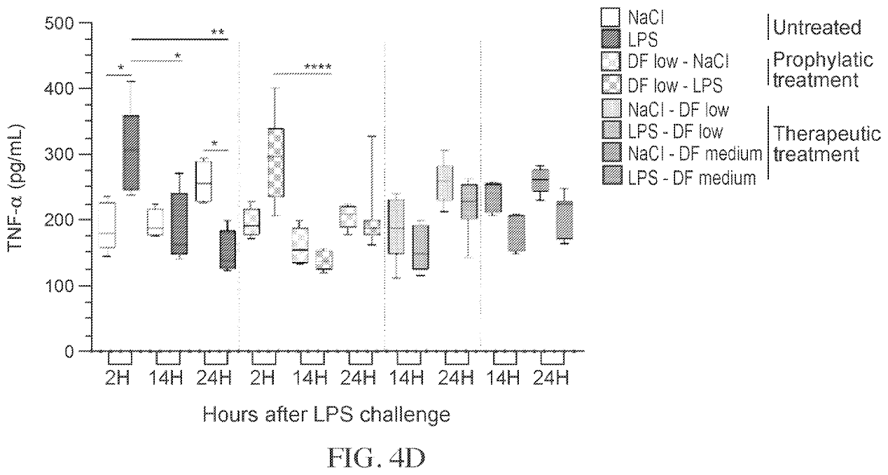

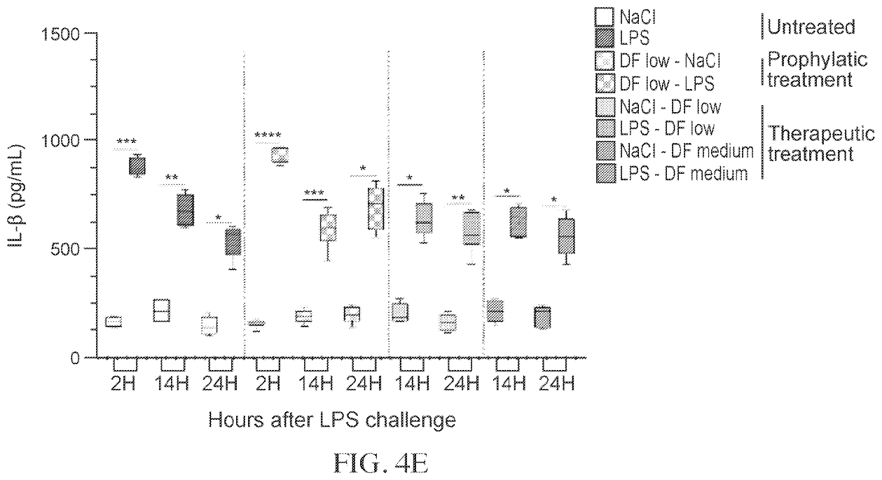

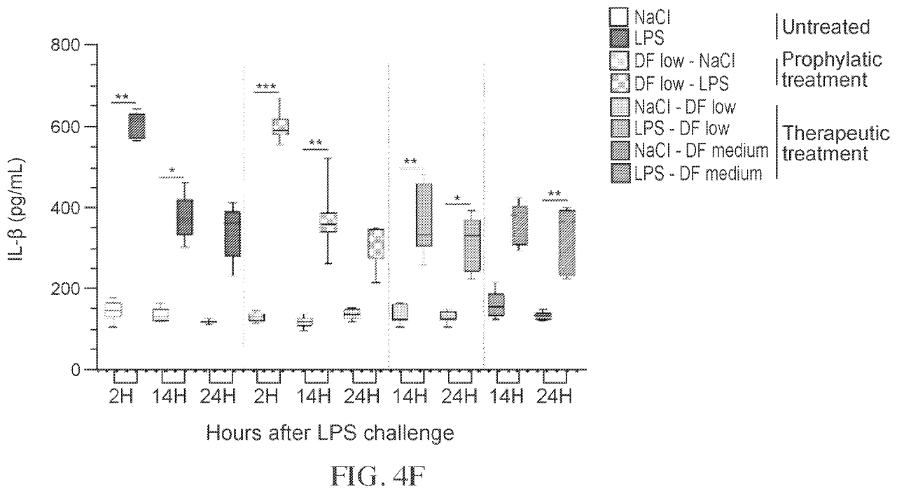

LPS induced a significant gain in lung weight compared with NaCl group 14 hours after LPS challenge (181.3±5.0 mg vs 148.8±7.6 mg, respectively (*p=0.033) (FIG. 4A) but did not affect liver weight (FIG. 4B). In lung and liver, TNF-α and IL-1β production was peaking at 2 hours after LPS challenge and decreased with time but remaining high at 24 hours (FIGS. 4C, 4D, 4E & 4F). Prophylactic and therapeutic treatment with defibrotide had no impact on TNF-α and IL-1β productions in liver and lung, respectively. Defibrotide in prophylaxis had a trend to remain a high level of IL-1β production in lungs of LPS mice compared with LPS mice no treated at 24 hours after LPS challenge (p=0.085).

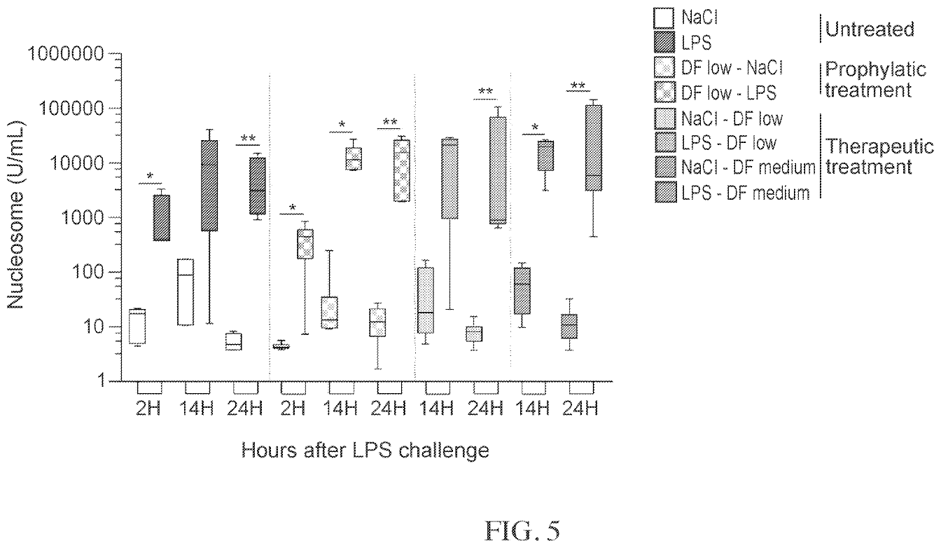

Next, the effects of defibrotide on nucleosome release were studied (FIG. 5A). Endotoxemia induced significant nucleosome release early in inflammatory response 2 hours after LPS challenge (*p=0.016), and still high 24 hours after LPS-induced sepsis (**p=0.0062) compared with control groups. The course of nucleosome release seemed to decline at 24 hours after LPS challenge in LPS untreated as well as LPS treated in therapeutic, whereas in LPS treated in prophylaxis the nucleosome levels seemed to remain at high level (not significant).

In conclusion, prophylactic defibrotide administration improved mortality in LPS-induced sepsis and resulted in a quicker improvement of renal function than mice receiving LPS only in the absence of defibrotide. The inflammatory response as reflected by the production of TNF-α, IL-1β, IL-6, KC and MPO in the spleen and by the production of TNF-α and IL-1β in spleen and liver, respectively was not dampened by defibrotide administration. Prophylactic defibrotide administration resulted in a sustained TNF-α and IL-1β production after 24 hours in spleen and liver, respectively. Finally, no effect of defibrotide treatment on nucleosome levels as a surrogate marker of systemic inflammation being increased after LPS challenge could be observed.

In the LPS-induced inflammatory model, prophylactic treatment with defibrotide at low dose, 25 mg/kg/d improved survival, improved renal function and interestingly prolonged the inflammatory response in the spleen and in lungs. Defibrotide had no impact on inflammatory response in liver. This is the first time that a study reveals a pro-inflammatory activity of defibrotide. Previously, several studies have reported the anti-inflammatory effect of defibrotide. In vitro, defibrotide induced a dose-dependent anti-inflammatory response of dendritic cells with a decrease of TNF-α and IL-12 productions and an increase IL-10 production (Francischetti et al. 2012). In a mouse model of cerebral malaria, defibrotide inhibited IFN-gamma in the blood (Francischetti et al. 2012). A profiling study demonstrated the impact of defibrotide on anti-inflammatory response of endothelial cells exposed to LPS (Orlando et al. 2020). In addition, defibrotide had no impact on circulating nucleosome which is an established marker for systemic inflammation.

Prophylactic defibrotide treatment has been studied in prophylaxis clinical trials study in children after hematopoietic stem cell transplantation to prevent the occurrence of veno-occlusive disease/severe sinusoidal obstructive syndrome (VOD/SOS) in high-risk pediatric patients (Karagun et al. 2022). Despite several positive clinical studies (Cappelli et al. 2009; Corbacioglu et al. 2012; Pichler et al. 2017; Corbacioglu et al. 2020), the use of defibrotide as a prophylactic agent for this respective indication has not been approved yet. But defibrotide has been approved by the FDA in the treatment of hepatic VOD. In an early clinical trial, patients with VOD received escalating doses of defibrotide from 10 mg/kg/day to a maximum of 100 mg/kg/day. The study shows that defibrotide at high doses >100 mg/kg/day were associated with increased toxicity (Triplett et al. 2015). The instant LPS-induced sepsis preclinical model confirms this toxicity aspect of defibrotide at high dose with a deleterious effect on survival at 175 mg/kg/day or 350 mg/kg/day by intraperitoneal injection.

Therapeutic administration of defibrotide at usual dose in the LPS-induced sepsis model showed a trend in improvement of survival rate but did not display any effect on the inflammatory response assessed in the spleen, liver and lung, respectively. In the clinical setting a continuous infusion of defibrotide could be applied due to the short half-life of defibrotide ranges from 9.8 to 21.1 minutes from doses at 0.5 mg/kg and 16 mg/kg respectively (Noseda, Fragiacomo, et Ferrari 1986). Intraperitoneal injections every 8 hours may on one hand induce potential toxic peak concentrations, on the other hand may result in drug levels beyond the therapeutic range.

The study showed a positive effect of defibrotide on renal function in the in the LPS-induced sepsis model. In preclinical ischemia models, defibrotide provided relevant protection against renal injury (Comandella et al. 1993; Aydemir et al. 2003; abstract Pine at al. 2021). Histones are mediator of cell death through TLR-2 and TLR-4 (Xu et al. 2011). A recent study demonstrated that histones were cytotoxic to renal endothelial cells and tubular epithelial cells in vitro (Allam et al. 2012). In vivo, neutralization of histone H4 or citrullinated histone H3 was shown to significantly improve survival in a CLP mouse model (Xu et al. 2011) or endotoxic mouse model (Deng et al. 2020). Considering histones to induce or mediate kidney damage on a molecular level, defibrotide may neutralize histone cytotoxicity, since double stranded cell-free DNA has been shown to neutralize histone cytotoxicity (Marsman et al. 2017).

### Example 2—Efficacy Study for Defibrotide Administered Intravenously Four Times Daily to Wistar Rats with LPS-induced Sepsis

The objectives of the study were to determine the efficacy of defibrotide to mitigate the acute sepsis-related inflammatory reaction, endothelial cell activation, coagulopathy, and lung injury following a 2-hour intravenous infusion 4 times to male Wistar rats.

Ten (10) male Wistar rats per group were given the reference item (saline) or the test item, Defibrotide at doses of 20, 40 and 80 mg/kg/dose as a 2-hour infusion, 6 hours apart, starting 5 minutes after initiation of LPS infusion (1st dose only) for sepsis induction. In addition, two groups were not administered LPS, one (n=10) received 80 mg/kg/dose defibrotide while the 2nd received saline (n=5), another group received LPS but was not treated.

During this study, assessments included mortality checks, clinical observations, body weights, body temperatures and clinical pathology evaluation (coagulation and clinical chemistry). Blood samples for cytokine, toxicokinetic and biomarker analysis were collected at targeted time points. Necropsy was performed on all surviving animals 24 hours post LPS-challenge as well as from pre-terminal animals at time of death or euthanasia. The left lung was used for bronchoalveolar fluid collection for cytokine analysis while the right lung was collected for Wet-to-Dry ratio measurement (6/10 animals) or retained in formalin for histological evaluation (4/10 animals).

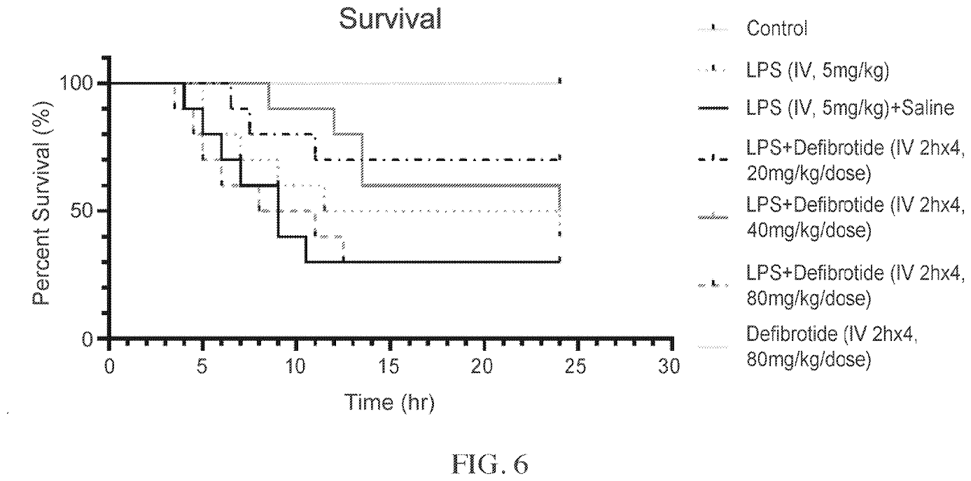

FIG. 6 shows that four consecutive 2-hour intravenous infusions (6 hours apart) of defibrotide at 20 mg/kg/dose to LPS-challenged rats resulted in 40% more animals surviving to 24 hours compared to controls. A similar mortality rate was observed between animals treated with defibrotide at the 80 mg/kg/dose and controls. There were no differences in onset or severity of LPS-related clinical signs, or effects on body temperatures, coagulation, clinical chemistry or lung water content between LPS-challenged animals given defibrotide and LPS-challenged control animals. Macroscopic findings at necropsy were consistent with sepsis-induced inflammatory reaction and were generally comparable across LPS-challenged groups. Microscopic examination of the right lung showed minimal to mild hemorrhage, minimal to mild mixed inflammation, and minimal to moderate intravascular leukocytic aggregates in LPS-challenged animals with generally no difference between defibrotide and control-treated groups.

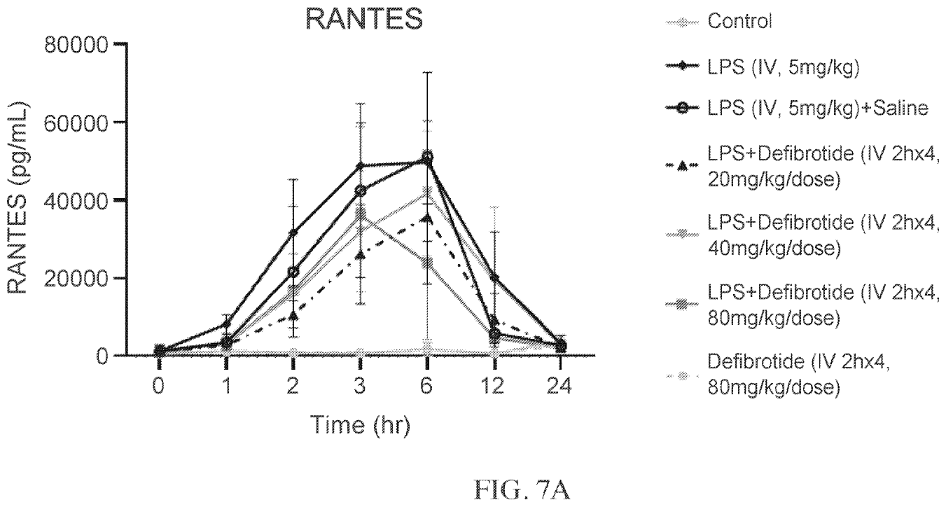

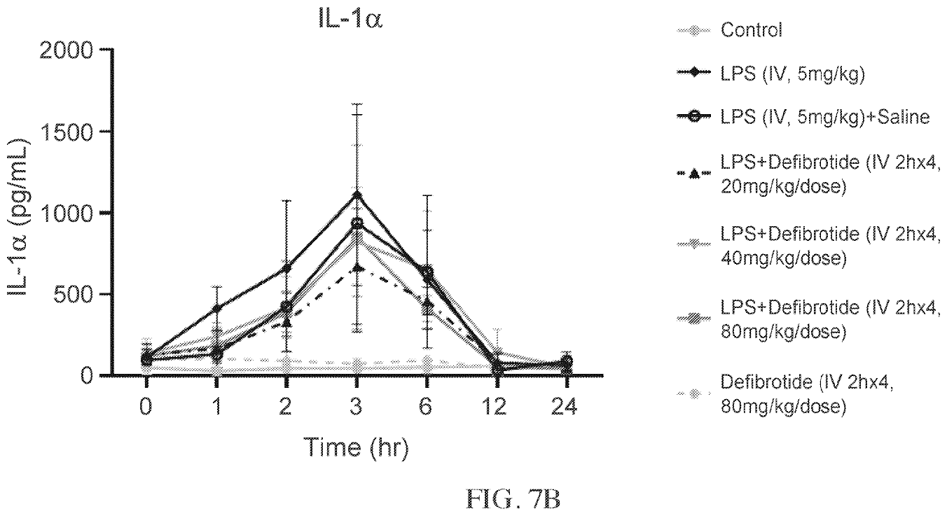

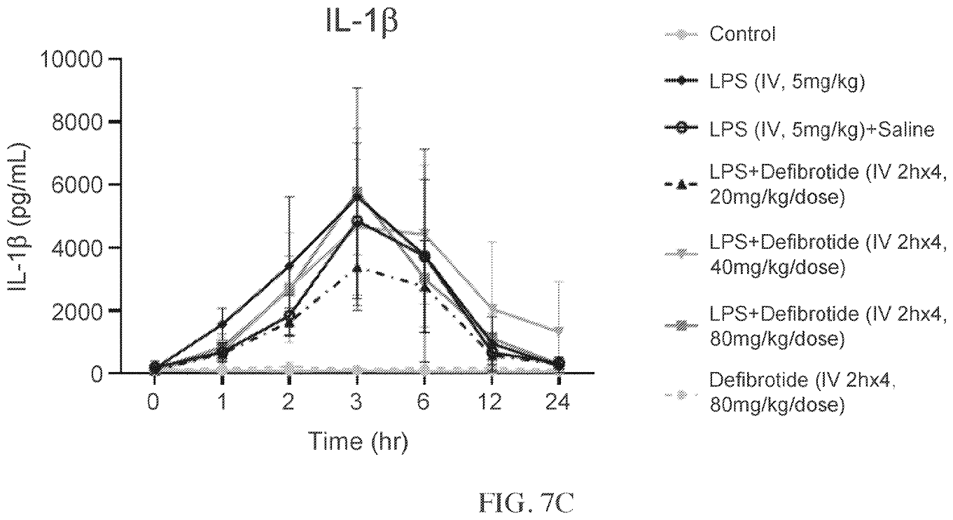

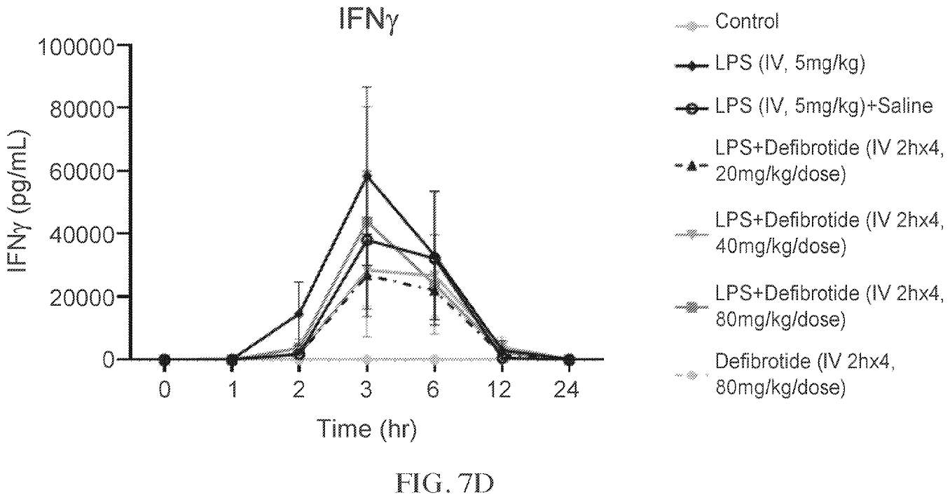

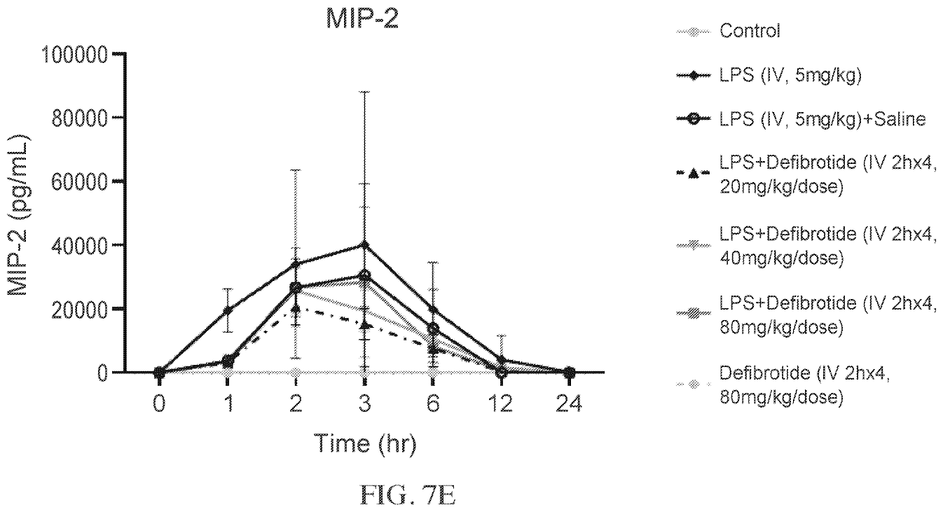

FIGS. 7A-7E shows plasma expression of RANTES (FIG. 7A), IL-1α (FIG. 7B), IL-1β (FIG. 7C), IFNγ (FIG. 7D), and MIP-2 (FIG. 7E) after LPS administration following saline or defibrotide 2-hour intravenous infusion 20 mg/kg, 40 mg/kg and 80 mg/kg/dose in LPS-challenged rats. Defibrotide administration at the low dose (20 mg/kg/dose) was associated with a delayed onset and/or reduced plasma levels of cytokines and chemokines, including IFNγ, IL-1α, IL-1β, RANTES, and MIP-2 compared to LPS control.

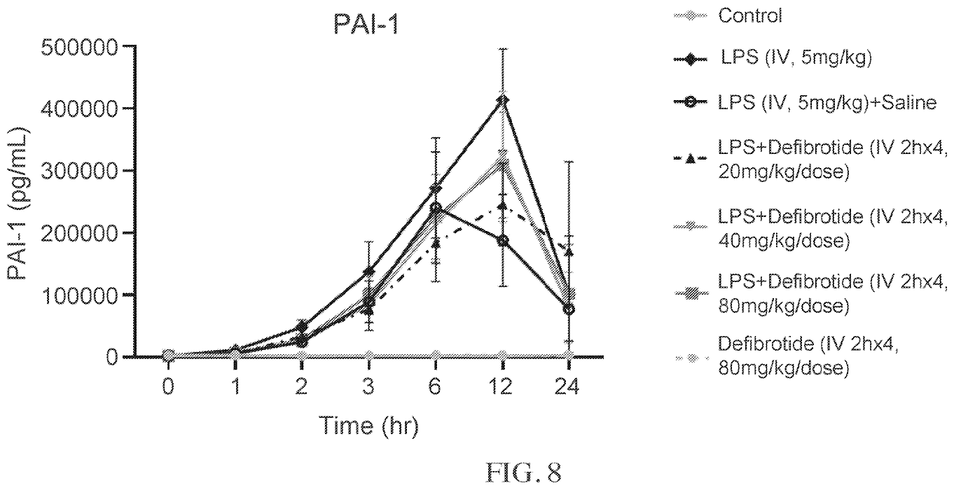

FIG. 8 shows the plasma biomarker expression after LPS administration following saline and defibrotide. The results showed that defibrotide administration at the low dose (20 mg/kg/dose) reduced the plasma level of the coagulation biomarker PAI-1.

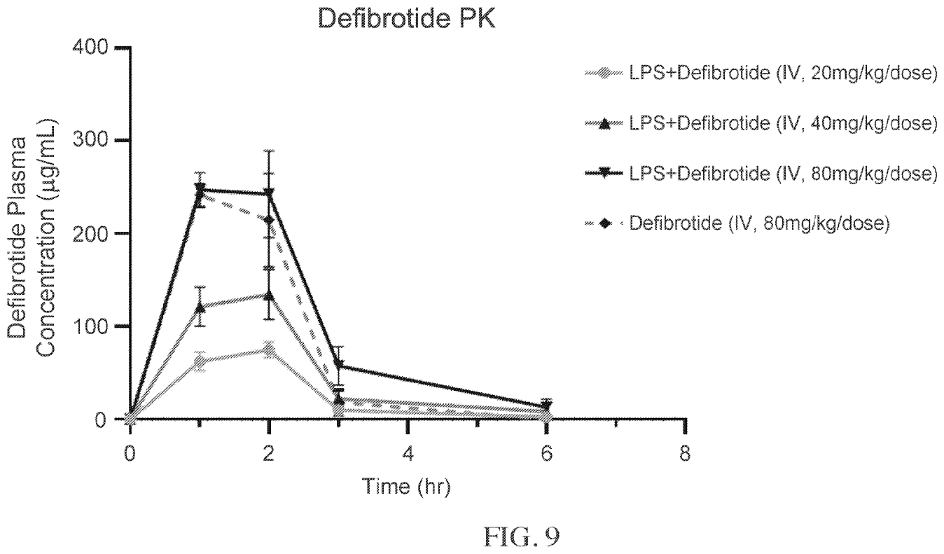

FIG. 9 shows the defibrotide PK profile in rats. The results showed dose proportional changes in circulating levels of defibrotide. Following the first 2-hour IV infusion of 20 mg/kg, 40 mg/kg and 80 mg/kg defibrotide dose in LPS challenged rats, defibrotide plasma exposures (Cmax and AUC) increased in an approximate dose proportional manner. Defibrotide Cmax was 74.7, 136 and 257 μg/mL for 20 mg/kg, 40 mg/kg and 80 mg/kg defibrotide dose levels, respectively. Defibrotide AUC₀₋₆ was 157, 312 and 612 h*μg/mL for 20 mg/kg, 40 mg/kg and 80 mg/kg defibrotide dose levels, respectively.

Overall, data collected under the condition of this study demonstrated that four consecutive 2-hour intravenous infusions (6 hours apart) of defibrotide at 20 mg/kg/dose improved sepsis-related mortality in rats. Defibrotide administration at the low lose (20 mg/kg/dose) was associated with a delayed onset and/or reduced plasma levels of cytokines and chemokines, including IFNγ, IL-1α, IL-1β, RANTES, and MIP-2 and the coagulation biomarker PAI-1 compared to LPS control. Defibrotide PK profile in rats showed dose proportional changes in circulating levels of defibrotide.

### REFERENCES

1. Ali, Ramadan A., Shanea K. Estes, Alex A. Gandhi, Srilakshmi Yalavarthi, Claire K. Hoy, Hui Shi, Yu Zuo, Doruk Erkan, et Jason S. Knight. 2021. «Defibrotide Inhibits Antiphospholipid Antibody-Mediated NET Formation and Venous Thrombosis». Arthritis & Rheumatology (Hoboken, N.J.), novembre. https://doi.org/10.1002/art.42017.
2. Allam, Ramanjaneyulu, Christina Rebecca Scherbaum, Murthy Narayana Darisipudi, Shrikant R. Mulay, Holger Hägele, Julia Lichtnekert, Jan Henrik Hagemann, et al. 2012. «Histones from Dying Renal Cells Aggravate Kidney Injury via TLR2 and TLR4». Journal of the American Society of Nephrology: JASN 23 (8): 1375 88.https://doi.org/10.1681/ASN.2011111077
3. Andreasen, A. S., K. S. Krabbe, R. Krogh-Madsen, S. Taudorf, B. K. Pedersen, et K. Moller. 2008. «Human Endotoxemia as a Model of Systemic Inflammation». Current Medicinal Chemistry 15 (17): 1697 1705. https://doi.org/10.2174/092986708784872393.
4. Aydemir, E. O., A. Var, B. S. Uyanik, O. Ilkgül, H. Aydede, et A. Sakarya. 2003. «The Protective Mechanisms of Defibrotide on Liver Ischaemia-Reperfusion Injury». Cell Biochemistry and Function 21 (4): 307 10. https://doi.org/10.1002/cbf.1034.
5. Bahoush, Golamreza, et Maryam Vafapour. 2020. «A Case Report of Severe Veno-Occlusive Disease Following Autologous Stem Cell Transplantation Successfully Treated with Defibrotide». European Journal of Translational Myology 30 (3): 9161. https://doi.org/10.4081/ejtm.2020.9161.
6. Brinkmann, Volker, Ulrike Reichard, Christian Goosmann, Beatrix Fauler, Yvonne Uhlemann, David S. Weiss, Yvette Weinrauch, et Arturo Zychlinsky. 2004. «Neutrophil Extracellular Traps Kill Bacteria». Science (New York, N.Y.) 303 (5663): 1532 35. https://doi.org/10.1126/science.1092385.
7. Cappelli, Barbara, Robert Chiesa, Costanza Evangelio, Alessandra Biffi, Tito Roccia, Ilaria Frugnoli, Erika Biral, et al. 2009. «Absence of VOD in Paediatric Thalassaemic HSCT Recipients Using Defibrotide Prophylaxis and Intravenous Busulphan». British Journal of Haematology 147 (4): 554 60. https://doi.org/10.1111/j.1365-2141.2009.07871.x.
8. Comandella, M. G., P. Rigotti, M. Valente, G. Pittoni, N. Baldan, E. Ganz, P. Amodio, et E. Ancona. 1993. «Functional and Morphological Effects of Defibrotide on Renal Ischemia». Research in Experimental Medicine. Zeitschrift Fur Die Gesamte Experimentelle Medizin Einschliesslich Experimenteller Chirurgie 193 (2): 65 71. https://doi.org/10.1007/bf02576212.
9. Copeland, Shannon, H. Shaw Warren, Stephen F. Lowry, Steve E. Calvano, Daniel Remick, et Inflammation and the Host Response to Injury Investigators. 2005. «Acute Inflammatory Response to Endotoxin in Mice and Humans». Clinical and Diagnostic Laboratory Immunology 12 (1): 60 67. https://doi.org/10.1128/CDLI.12.1.60-67.2005.
10. Corbacioglu, Selim, Simone Cesaro, Maura Faraci, Dominique Valteau-Couanet, Bernd Gruhn, Attilio Rovelli, Jaap J. Boelens, et al. 2012. «Defibrotide for Prophylaxis of Hepatic Veno-Occlusive Disease in Paediatric Haemopoietic Stem-Cell Transplantation: An Open-Label, Phase 3, Randomised Controlled Trial». Lancet (London, England) 379 (9823): 1301 9. https://doi.org/10.1016/S0140-6736(11)61938-7.
11. Corbacioglu, Selim, Saurabh Aggarwal, Ozlem Topaloglu, et William Tappe. 2020. «A Systematic Review of Studies of Defibrotide Prophylaxis for Veno-Occlusive Disease/Sinusoidal Obstruction Syndrome». Biology of Blood and Marrow Transplantation 26 (3, Supplement): S133. https://doi.org/10.1016/j.bbmt.2019.12.655.
12. Czaikoski, Paula Giselle, José Maurício Segundo Correia Mota, Daniele Carvalho Nascimento, Fabiane Sônego, Fernanda Vargas e Silva Castanheira, Paulo Henrique Melo, Gabriela Trentin Scortegagna, et al. 2016. «Neutrophil Extracellular Traps Induce Organ Damage during Experimental and Clinical Sepsis». PLoS ONE 11 (2): e0148142. https://doi.org/10.1371/journal.pone.0148142.
13. David, S., et F. M. Brunkhorst. 2017. «[Sepsis-3: What has been confirmed in therapy?]». Der Internist 58 (12): 1264 71. https://doi.org/10.1007/s00108-017-0338-5.
14. Deng, Qiufang, Baihong Pan, Hasan B. Alam, Yingjian Liang, Zhenyu Wu, Baoling Liu, Nirit Mor-Vaknin, et al. 2020. «Citrullinated Histone H3 as a Therapeutic Target for Endotoxic Shock in Mice». Frontiers in Immunology 10 (janvier): 2957. https://doi.org/10.3389/fimmu.2019.02957.
15. Denning, Naomi-Liza, Monowar Aziz, Steven D. Gurien, et Ping Wang. 2019. «DAMPs and NETs in Sepsis». Frontiers in Immunology 10 (octobre). https://doi.org/10.3389/fimmu.2019.02536.
16. Ferraresso, M., P. Rigotti, S. M. Stepkowski, T. C. Chou, et B. D. Kahan. 1993. «Immunosuppressive Effects of Defibrotide». Transplantation 56 (4): 928 33. https://doi.org/10.1097/00007890-199310000-00029.
17. Fleischmann, Carolin, André Scherag, Neill K. J. Adhikari, Christiane S. Hartog, Thomas Tsaganos, Peter Schlattmann, Derek C. Angus, Konrad Reinhart, et International Forum of Acute Care Trialists. 2016. «Assessment of Global Incidence and Mortality of Hospital-Treated Sepsis. Current Estimates and Limitations». American Journal of Respiratory and Critical Care Medicine 193 (3): 259 72. https://doi.org/10.1164/rccm.201504-07810C.
18. Francischetti, Ivo M. B., Carlo J. Oliveira, Graciela R. Ostera, Stephanie B. Yager, Françoise Debierre-Grockiego, Vanessa Carregaro, Giovanna Jaramillo-Gutierrez, et al. 2012. «Defibrotide interferes with several steps of the coagulation-inflammation cycle and exhibits therapeutic potential to treat severe malaria». Arteriosclerosis, Thrombosis, and Vascular Biology 32 (3): 786 98. https://doi.org/10.1161/ATVBAHA.111.240291.
19. Hamaguchi, Shigeto, Yukihiro Akeda, Norihisa Yamamoto, Masafumi Seki, Kouji Yamamoto, Kazunori Oishi, et Kazunori Tomono. 2015. «Origin of Circulating Free DNA in Sepsis: Analysis of the CLP Mouse Model». Mediators of Inflammation 2015 (juillet): e614518. https://doi.org/10.1155/2015/614518.
20. Huang, Min, Shaoli Cai, et Jingqian Su. 2019. «The Pathogenesis of Sepsis and Potential Therapeutic Targets». International Journal of Molecular Sciences 20 (21): 5376. https://doi.org/10.3390/ijms20215376.
21. Karagun, Barbaros S., Tuana Akbas, Fatih Erbey, İlgen Sasmaz, et Bulent Antmen. 2022. «The Prophylaxis of Hepatic Veno-Occlusive Disease/Sinusoidal Obstruction Syndrome With Defibrotide After Hematopoietic Stem Cell Transplantation in Children: Single Center Experience». Journal of Pediatric Hematology/Oncology 44 (1): e35. https://doi.org/10.1097/MPH.0000000000002379.
22. Lankelma, Jacqueline M., Duncan R. Cranendonk, Clara Belzer, Alex F. de Vos, Willem M. de Vos, Tom van der Poll, et W. Joost Wiersinga. 2017. «Antibiotic-Induced Gut Microbiota Disruption during Human Endotoxemia: A Randomised Controlled Study». Gut 66 (9): 1623 30. https://doi.org/10.1136/gutjnl-2016-312132
23. Marsman, Gerben, Helen von Richthofen, Ingrid Bulder, Florea Lupu, Jan Hazelzet, Brenda M. Luken, et Sacha Zeerleder. 2017. «DNA and Factor VII-Activating Protease Protect against the Cytotoxicity of Histones». Blood Advances 1 (26): 2491 2502. https://doi.org/10.1182/bloodadvances.2017010959.
24. Meer, Anne Jan van der, Anna Kroeze, Arie J. Hoogendijk, Aicha Ait Soussan, C. Ellen van der Schoot, Walter A. Wuillemin, Carlijn Voermans, Tom van der Poll, et Sacha Zeerleder. 2019. «Systemic Inflammation Induces Release of Cell-Free DNA from Hematopoietic and Parenchymal Cells in Mice and Humans». Blood Advances 3 (5): 724 28. https://doi.org/10.1182/bloodadvances.2018018895.
25. Messer, Juerg. 2015. «Improving intravenous injection in black mice», janvier.
26. Montfoort, Maurits L. van, Femke Stephan, Mandy N. Lauw, Barbara A. Hutten, Gerard J. Van Mierlo, Shabnam Solati, Saskia Middeldorp, Joost C. M. Meijers, et Sacha Zeerleder. 2013. «Circulating Nucleosomes and Neutrophil Activation as Risk Factors for Deep Vein Thrombosis». Arteriosclerosis, Thrombosis, and Vascular Biology 33 (1): 147 51. https://doi.org/10.1161/ATVBAHA.112.300498.
27. Niada, R., M. Mantovani, G. Prino, R. Pescador, R. Porta, F. Berti, G. C. Folco, C.

Omini, et T. Viganò. 1982. «PGI 2-Generation and Antithrombotic Activity of Orally Administered Defibrotide ». Pharmacological Research Communications 14 (10): 949 57. https://doi.org/10.1016/s0031-6989(82)80059-3.
28. Niedźwiedzka-Rystwej, Paulina, Weronika Repka, Beata Tokarz-Deptuła, et Wiesław Deptuła. 2019. «“In sickness and in health”—how neutrophil extracellular trap (NET) works in infections, selected diseases and pregnancy». Journal of Inflammation 16 (1): 15. https://doi.org/10.1186/s 12950-019-0222-2.
29. Noseda, G., C. Fragiacomo, et D. Ferrari. 1986. «Pharmacokinetics of Defibrotide in Healthy Volunteers». Haemostasis 16 Suppl 1:26 30. https://doi.org/10.1159/000215336
30. Orlando, Nicoletta, Gabriele Babini, Patrizia Chiusolo, Caterina Giovanna Valentini, Valerio De Stefano, et Luciana Teofili. 2020. «Pre-Exposure to Defibrotide Prevents Endothelial Cell Activation by Lipopolysaccharide: An Ingenuity Pathway Analysis». Frontiers in Immunology 11 (décembre): 585519. https://doi.org/10.3389/fimmu.2020.585519.
31. Papayannopoulos, Venizelos. 2018. «Neutrophil Extracellular Traps in Immunity and Disease». Nature Reviews. Immunology 18 (2): 134 47. https://doi.org/10.1038/nri.2017.105
32. Pescador, R., M. Mantovani, G. Prino, et M. Madonna. 1983. «Pharmacokinetics of Defibrotide and of Its Profibrinolytic Activity in the Rabbit». Thrombosis Research 30 (1): 1 11 .
33. Pescador, R., L. Capuzzi, M. Mantovani, A. Fulgenzi, et M. E. Ferrero. 2013. «Defibrotide: Properties and clinical use of an old/new drug». Vascular Pharmacology 59 (1): 1 10. https://doi.org/10.1016/j.vph.2013.05.001.
34. Pichler, Herbert, Karolina Horner, Gernot Engstler, Ulrike Poetschger, Evgenia Glogova, Susanne Karlhuber, Manuel Martin, et al. 2017. «Cost-Effectiveness of Defibrotide in the Prophylaxis of Veno-Occlusive Disease after Pediatric Allogeneic Stem Cell Transplantation». Biology of Blood and Marrow Transplantation 23 (7): 1128 33. https://doi.org/10.1016/j. bbmt.2017.03.022.
35. Pine P, Z. Sun, A. MAtas, J. Shi, A. Ebrahimnejad, M. Zanette, P. Halloran, J. S. Bromberg, et R. Hanvesakul. «Effect of Defibrotide on Kidney Ischemia Reperfusion Injury in a Preclinical Renal Transplant Model». s. d. ATC Abstracts (blog). https://atcmeetingabstracts.com/abstract/effect-of-defibrotide-on-kidney-ischemia-reperfusion-injury-in-a-preclinical-renal-transplant-model/.
36. Shi, Hui, Alex A. Gandhi, Stephanie A. Smith, Diane Chiang, Srilakshmi Yalavarthi, Ramadan A. Ali, Chao Liu, et al. 2021. «Endothelium-protective, histone-neutralizing properties of the polyanionic agent defibrotide». medRxiv, février. https://doi.org/10.1101/2021.02.21.21252160.
37. Raymond, Steven L., David C. Holden, Juan C. Mira, Julie A. Stortz, Tyler J. Loftus, Alicia M. Mohr, Lyle L. Moldawer, Frederick A. Moore, Shawn D. Larson, et Philip A. Efron. 2017. «Microbial Recognition and Danger Signals in Sepsis and Trauma». Biochimica et biophysica acta 1863 (10 Pt B): 2564 73. https://doi.org/10.1016/j.bbadis.2017.01.013.
38. Richardson, Paul G., Selim Corbacioglu, Vincent Trien-Vinh Ho, Nancy A. Kernan, Leslie Lehmann, Craig Maguire, Michelle Maglio, et al. 2013. «Drug safety evaluation of defibrotide». Expert Opinion on Drug Safety 12 (1): 123 36. https://doi.org/10.1517/14740338.2012.749855.
39. Richardson, Paul G., Enric Carreras, Massimo Iacobelli, et Bijan Nejadnik. 2018. «The use of defibrotide in blood and marrow transplantation». Blood Advances 2 (12): 1495 1509. https://doi.org/10.1182/bloodadvances.2017008375.
40. Saffarzadeh, Mona, Christiane Juenemann, Markus A. Queisser, Guenter Lochnit, Guillermo Barreto, Sebastian P. Galuska, Juergen Lohmeyer, et Klaus T. Preissner. 2012. «Neutrophil Extracellular Traps Directly Induce Epithelial and Endothelial Cell Death: A Predominant Role of Histones». PloS One 7 (2): e32366. https://doi.org/10.1371/journal.pone.0032366.
41. Schoergenhofer, Christian, Nina Buchtele, Georg Gelbenegger, Ulla Derhaschnig, Christa Firbas, Katarina D. Kovacevic, Michael Schwameis, Philipp Wohlfarth, Werner Rabitsch, et Bernd Jilma. 2019. «Defibrotide Enhances Fibrinolysis in Human Endotoxemia—a Randomized, Double Blind, Crossover Trial in Healthy Volunteers». Scientific Reports 9 (1): 11136. https://doi.org/10.1038/s41598-019-47630-6.
42. Schönrich, Günther, et Martin J. Raftery. 2016. «Neutrophil Extracellular Traps Go Viral ». Frontiers in Immunology 7 (septembre): 366. https://doi.org/10.3389/fimmu.2016.00366.
43. Shi, Hui, Alex A. Gandhi, Stephanie A. Smith, Qiuyu Wang, Diane Chiang, Srilakshmi Yalavarthi, Ramadan A. Ali, et al. s. d. «Endothelium-protective, histone-neutralizing properties of the polyanionic agent defibrotide». JCI Insight 6 (17): e149149. https://doi.org/10.1172/jci.insight.149149.
44. Shrum, Bradly, Ram V. Anantha, Stacey X. Xu, Marisa Donnelly, S. M. Mansour Haeryfar, John K. McCormick, et Tina Mele. 2014. «A Robust Scoring System to Evaluate Sepsis Severity in an Animal Model». BMC Research Notes 7 (avril): 233. https://doi.org/10.1186/1756-0500-7-233.
45. Triplett, Brandon M., Hani I. Kuttab, Guolian Kang, et Wing Leung. 2015. «Escalation to High Dose Defibrotide in Patients with Hepatic Veno-Occlusive Disease». Biology of blood and marrow transplantation: journal of the American Society for Blood and Marrow Transplantation 21 (12): 2148 53. https://doi.org/10.1016/j.bbmt.2015.08.013.
46. Wang, Shanping, Keai Sinn Tan, Huimin Beng, Fei Liu, Jiandong Huang, Yihe Kuai, Rui Zhang, et Wen Tan. 2021. «Protective Effect of Isosteviol Sodium against LPS-Induced Multiple Organ Injury by Regulating of Glycerophospholipid Metabolism and Reducing Macrophage-Driven Inflammation». Pharmacological Research 172 (octobre): 105781. https://doi.org/10.1016/j.phrs.2021.105781.
47. Xu, Jun, Xiaomei Zhang, Rosana Pelayo, Marc Monestier, Concetta T. Ammollo, Fabrizio Semeraro, Fletcher B. Taylor, Naomi Esmon, Florea Lupu, et Charles T. Esmon. 2009. «Extracellular histones are major mediators of death in sepsis ». Nature medicine 15 (11): 1318 21. https://doi.org/10.1038/nm.2053.
48. Xu, Jun, Xiaomei Zhang, Marc Monestier, Naomi L. Esmon, et Charles T. Esmon. 2011. «Extracellular Histones Are Mediators of Death through TLR2 and TLR4 in Mouse Fatal Liver Injury». Journal of Immunology (Baltimore, Md.: 1950) 187 (5): 2626 31. https://doi.org/10.4049/jimmunol.1003930.
49. Zeerleder, Sacha, Femke Stephan, Marieke Emonts, Ester D. de Kleijn, Charles T. Esmon, Katalin Varadi, Cornelis Erik Hack, et Jan A. Hazelzet. 2012. «Circulating Nucleosomes and Severity of Illness in Children Suffering from Meningococcal Sepsis Treated with Protein C». Critical Care Medicine 40 (12): 3224 29. https://doi.org/10.1097/CCM.0b013e318265695f.
50. Zeerleder, Sacha, Bas Zwart, Henk te Velthuis, Rishi Manoe, Ingrid Bulder, Irma Rensink, et Lucien A. Aarden. 2007. «A Plasma Nucleosome Releasing Factor (NRF) with Serine Protease Activity Is Instrumental in Removal of Nucleosomes from Secondary Necrotic Cells». FEBS Letters 581 (28): 5382 88. https://doi.org/10.1016/j.febslet.2007.10.037.
51. Zeerleder, Sacha, Bas Zwart, Walter Wuillemin, Lucien Aarden, A. B. Groeneveld, Christoph Caliezi, Annemarie van Nieuwenhuijze, et al. 2003. «Elevated Nucleosome Levels in Systemic Inflammation and Sepsis*». Critical Care Medicine 31 (7): 1947 51. https://doi.org/10.1097/01.CCM.0000074719.40109.95.
52. Zeerleder, Sacha, Christoph Caliezi, Gerard van Mierlo, Anke Eerenberg-Belmer, Irmela Sulzer, C. Erik Hack, et Walter A. Wuillemin. 2003. «Administration of C1 Inhibitor Reduces Neutrophil Activation in Patients with Sepsis». Clinical and Diagnostic Laboratory Immunology 10 (4): 529 35. https://doi.org/10.1128/CDLI.10.4.529-535.2003.

What is claimed:

1. A method of preventing or treating sepsis comprising administering a prophylactically effective amount of defibrotide to a subject in need thereof.

2. The method of claim 1, wherein the subject is suffering from an infection.

3. The method of any one of claims 1-2, wherein the administration is before the onset of sepsis or symptoms thereof.

4. The method of any one of claims 1-3, wherein the subject is at high risk for developing sepsis.

5. The method of claim 4, wherein the subject is selected from a group consisting of an adult 65 or older, a child younger than one, a person with a chronic condition, a person with a weakened immune system, and a pregnant woman.

6. The method of claim 5, wherein the chronic condition comprises diabetes, cancer, lung disease, kidney disease, and liver disease.

7. The method of any one of claims 1-6, wherein an altered level of a biomarker associated with sepsis is detected in the subject.

8. The method of claim 7, wherein the biomarker is selected from a group consisting of IFNγ, IL-1α, IL-1β, RANTES, MIP-2 and PAI-1.

9. The method of any one of claims 1-8, wherein the administration of defibrotide continues until symptoms improve.

10. The method of any one of claims 1-9, wherein the defibrotide is administered at a dose between 1 mg/kg and 40 mg/kg.

11. The method of claim 10, wherein the defibrotide is administered at a dose of 20 mg/kg.

12. The method any one of claims 1-11, wherein the defibrotide is administered once a day.

13. The method any one of claims 1-11, wherein the defibrotide is administered in multiple doses per day.

14. The method of claim 13, wherein the defibrotide is administered in two to ten doses per day.

15. The method of claim 14, wherein the defibrotide is administered four times a day.

16. The method of claim 14, wherein the defibrotide is administered every six hours.

17. The method any one of claims 1-11, wherein the defibrotide is administered by continuous infusion.

18. The method of any of claims 1-16, wherein the defibrotide formulation is formulated for subcutaneous delivery or intravenous delivery.

19. The method of claim 18, wherein the defibrotide is administered intravenously.

20. The method of claim 18, wherein the defibrotide is administered subcutaneously.

21. The method of claim 1-11, wherein the defibrotide is administered by four consecutive two-hour intravenous infusion at a dose of 20 mg/kg, six hours apart.
